SEQUENCE LISTING (20 sequences)

SEQ ID NO: 1 - DNA, 2662 bp, Homo sapiens
tcagtttgaaaaggaggatcgagctcactcgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccgccccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgcctgggcccaccacctgtccgtccctgtcccctttcctgctggg

SEQ ID NO: 2 - protein, 822 aa, Homo sapiens [sequence omitted]

SEQ ID NO: 3 - DNA, 3840 bp, Homo sapiens
ggccttccccctgcgaggatcgccgttggcccgggttggctttggaaagcggcggtggctttgggccgggctcggcctcgggaacgccaggggcccctgggtgcggacgggcgcggccaggagggggttaaggcgcaggcggcggcggggcgggggcgggcctggcgggcgccctctccgggccctttgttaacaggcgcgtcccggccagcggagacgcggccgccctgggcgggcgcgggcggcgggcggcggtgagggcggcctgcggggcggcgcccgggggccgggccgagccgggcctgagccgggcccggaccgagctgggagaggggctccggcccgatcgttcgcttggcgcaaaatgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccatgacagtggctgag

SEQ ID NO: 4 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 5 - DNA, 1461 bp, Homo sapiens
aggcggtgtttgtctgccggactgacgggcggccgggcggtgcgcggcggcggtggcggcggggaaaatggcggcgtcctccctggaacagaagctgtcccgcctggaagcaaagctgaagcaggagaaccgggaggcccggcggaggatcgacctcaacctggatatcagcccccagcggcccaggcccaccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacctgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaggagaagcacggtgtcatccaccgcgacgtcaagccctccaacatcctgctggacgagcggggccagatcaagttctgcgacttcggcatcagcggccgcctggtggactccaaagccaagacgcggagcgccggctgtgccgcctacatggcacccgagcgcattgaccccccagaccccaccaagccggactatgacatccgggccgacgtatggagcctgggcatctcgctggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtagctgcttggcggcggccagccccacagggggccaggggcatggccacaggcccccctccccacttggccacccagctgcctgccaggggagacctgggacctggacggccacctaggactgaggacagagagtgg

SEQ ID NO: 6 - protein, 419 aa, Homo sapiens [sequence omitted]

SEQ ID NO: 7 - DNA, 1050 bp, Homo sapiens
ggggggggggggcacttggcttcaaagctggctcttggaaattgagcggagacgagcggcttgttgtagctgccgtgcggccgccgcggaataataagccgggatctaccataccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatg

SEQ ID NO: 8 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 9 - DNA, 1480 bp, Homo sapiens
  misc_feature (104)..(106): n = a, c, g or t
gaattccgagcaagagcgcgggcgggtggcccaggcacgcagcgggtgaggaccgcgcccacagctcggcgccaaccaccgcgggcctcccagccagccccgcnnngagccgcagganccctggctgtggtcggggggcagtgggccatgctgggggcagtggaaggccccaggtggaagcaggcggaggacattagagacatctacgacttccgagatgttctgggcacgggggccttctcggaggtgatcctggcagaagataagaggacgcagaagctggtggccatcaaatgcattgccaaggaggccctggagggcaaggaaggcagcatggagaatgagattgctgtcctgcacaagatcaagcaccccaacattgtagccctggatgacatctatgagagtgggggccacctctacctcatcatgcagctggtgtcgggtggggagctctttgaccgtattgtggaaaaaggcttctacacggagcgggacgccagccgcctcatcttccaggtgctggatgctgtgaaatacctgcatgacctgggcattgtacaccgggatctcaagccagagaatctgctgtactacagcctggatgaagactccaaaatcatgatctccgactttggcctctccaagatggaggacccgggcagtgtgctctccaccgcctgtggaactccgggatacgtggcccctgaagtcctggcccagaagccctacagcaaggctgtggattgctggtccataggtgtcatcgcctacatcttgctctgcggttaccctcccttctatgacgagaatgatgccaaactctttgaacagattttgaaggccgagtacgagtttgactctccttactgggacgacatctctgactctgccaaagatttcatccggcacttgatggagaaggacccagagaaaagattcacctgtgagcaggccttgcagcacccatggattgcaggagatacagctctagataagaatatccaccagtcggtgagtgagcagatcaagaagaactttgccaagagcaagtggaagcaagccttcaatgccacggctgtggtgcggcacatgaggaaactgcagctgggcaccagccaggaggggcaggggcagacggcgagccatggggagctgctgacaccagtggctggggggccggcagctggctgttgctgtcgagactgctgcgtggagccgggcacagaactgtcccccacactgccccaccagctctagggccctggacctcgggtcatgatcctctgcgtgggagggcttgggggccagcctgctccccttccctccctgaaccgggagtttctctgccctgtcccctcctcacctgcttccctaccactcctcactgcattttccatacaaatgtttctattttattgttccttcttgtaataaagggaagataaaaccaaaaaaaaaaaaaaaaaaacggaattcc

SEQ ID NO: 10 - protein, 370 aa, Homo sapiens
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQEGQGQTASHGELLTPVAGGPAAGCCCRDCCVEPGTELSPTLPHQL

SEQ ID NO: 11 - DNA, 1782 bp, Homo sapiens
gggcgggcgagggatctgaaacttgcccacccttcgggatattgcaggacgctgcatcatgagcgacagtaaatgtgacagtcagttttatagtgtgcaagtggcagactcaaccttcactgtcctaaaacgttaccagcagctgaaaccaattggctctggggcccaagggattgtttgtgctgcatttgatacagttcttgggataagtgttgcagtcaagaaactaagccgtccttttcagaaccaaactcatgcaaagagagcttatcgtgaacttgtcctcttaaaatgtgtcaatcataaaaatataattagtttgttaaatgtgtttacaccacaaaaaactctagaagaatttcaagatgtgtatttggttatggaattaatggatgctaacttatgtcaggttattcacatggagctggatcatgaaagaatgtcctaccttctttaccagatgctttgtggtattaaacatctgcattcagctggtataattcatagagatttgaagcctagcaacattgttgtgaaatcagactgcaccctgaagatccttgactttggcctggcccggacagcgtgcactaacttcatgatgaccccttacgtggtgacacggtactaccgggcgcccgaagtcatcctgggtatgggctacaaagagaacgttgatatctggtcagtgggttgcatcatgggagagctggtgaaaggttgtgtgatattccaaggcactgaccatattgatcagtggaataaagttattgagcagctgggaacaccatcagcagagttcatgaagaaacttcagccaactgtgaggaattatgtcgaaaacagaccaaagtatcctggaatcaaatttgaagaactctttccagattggatattcccatcagaatctgagcgagacaaaataaaaacaagtcaagccagagatctgttatcaaaaatgttagtgattgatcctgacaagcggatctctgtagacgaagctctgcgtcacccatacatcactgtttggtatgaccccgccgaagcagaagccccaccacctcaaatttatgatgcccagttggaagaaagagaacatgcaattgaagaatggaaagagctaatttacaaagaagtcatggattgggaagaaagaagcaagaatggtgttgtaaaagatcagccttcagatgcagcagtaagtagcaacgccactccttctcagtcttcatcgatcaatgacatttcatccatgtccactgagcagacgctggcctcagacacagacagcagtcttgatgcctcgacgggaccccttgaaggctgtcgatgataggttagaaatagcaaacctgtcagcattgaaggaactctcacctccgtgggcctgaaatgcttgggagttgatggaaccaaatagaaaaactccatgttctgcatgtaagaaacacaatgccttgccctattcagacctgataggattgcctgcttagatgataaaatgaggcagaatatgtctgaagaaaaaaattgcaagccacacttctagagattttgttcaagatcatttcaggtgagcagttagagtaggtgaatttgtttcaaattgtactagtgacagtttctcatcatctgtaactgttgagatgtatgtgcatgtgaccacaaatgcttgcttggacttgcccatctagcactttggaaatcagtatttaaatgccaaataatcttccaggtagtgctgcttctgaagttatctcttaatcctcttaagtaatttgg

SEQ ID NO: 12 - protein, 424 aa, Homo sapiens [sequence omitted]

SEQ ID NO: 13 - DNA, 3668 bp, Homo sapiens
gtggtcttcccgcgcctgaggcggcggcggcaggagctgaggggagttgtagggaactgaggggagctgctgtgtcccccgcctcctcctccccatttccgggctcccgggaccatgtccgcgctggcgggtgaagatgtctggaggtgtccaggctgtggggaccacattgctccaagccagatatggtacaggactgtcaacgaaacctggcacggctcttgcttccggtgttcagaatgccaggattccctcaccaactggtactatgagaaggatgggaagctctactgccccaaggactactgggggaagtttggggagttctgtcatgggtgctccctgctgatgacagggccttttatggtggctggggagttcaagtaccacccagagtgctttgcctgtatgagctgcaaggtgatcattgaggatggggatgcatatgcactggtgcagcatgccaccctctactgtgggaagtgccacaatgaggtggtgctggcacccatgtttgagagactctccacagagtctgttcaggagcagctgccctactctgtcacgctcatctccatgccggccaccactgaaggcaggcggggcttctccgtgtccgtggagagtgcctgctccaactacgccaccactgtgcaagtgaaagaggtcaaccggatgcacatcagtcccaacaatcgaaacgccatccaccctggggaccgcatcctggagatcaatgggacccccgtccgcacacttcgagtggaggaggtggaggatgcaattagccagacgagccagacacttcagctgttgattgaacatgaccccgtctcccaacgcctggaccagctgcggctggaggcccggctcgctcctcacatgcagaatgccggacacccccacgccctcagcaccctggacaccaaggagaatctggaggggacactgaggagacgttccctaaggcgcagtaacagtatctccaagtcccctggccccagctccccaaaggagcccctgctgttcagccgtgacatcagccgctcagaatcccttcgttgttccagcagctattcacagcagatcttccggccctgtgacctaatccatggggaggtcctggggaagggcttctttgggcaggctatcaaggtgacacacaaagccacgggcaaagtgatggtcatgaaagagttaattcgatgtgatgaggagacccagaaaacttttctgactgaggtgaaagtgatgcgcagcctggaccaccccaatgtgctcaagttcattggtgtgctgtacaaggataagaagctgaacctgctgacagagtacattgaggggggcacactgaaggactttctgcgcagtatggatccgttcccctggcagcagaaggtcaggtttgccaaaggaatcgcctccggaatggcctatttgcactctatgtgcatcatccaccgggatctgaactcgcacaactgcctcatcaagttggacaagactgtggtggtggcagactttgggctgtcacggctcatagtggaagagaggaaaagggcccccatggagaaggccaccaccaagaaacgcaccttgcgcaagaacgaccgcaagaagcgctacacggtggtgggaaacccctactggatggcccctgagatgctgaacggaaagagctatgatgagacggtggatatcttctcctttgggatcgttctctgtgagatcattgggcaggtgtatgcagatcctgactgccttccccgaacactggactttggcctcaacgtgaagcttttctgggagaagtttgttcccacagattgtcccccggccttcttcccgctggccgccatctgctgcagactggagcctgagagcagaccagcattctcgaaattggaggactcctttgaggccctctccctgtacctgggggagctgggcatcccgctgcctgcagagctggaggagttggaccacactgtgagcatgcagtacggcctgacccgggactcacctccctagccctggcccagccccctgcaggggggtgttctacagccagcattgcccctctgtgccccattcctgctgtgagcagggccgtccgggcttcctgtggattggcggaatgtttagaagcagaacaagccattcctattacctccccaggaggcaagtgggcgcagcaccagggaaatgtatctccacaggttctggggcctagttactgtctgtaaatccaatacttgcctgaaagctgtgaagaagaaaaaaacccctggcctttgggccaggaggaatctgttactcgaatccacccaggaactccctggcagtggattgtgggaggctcttgcttacactaatcagcgtgacctggacctgctgggcaggatcccagggtgaacctgcctgtgaactctgaagtcactagtccagctgggtgcaggaggacttcaagtgtgtggacgaaagaaagactgatggctcaaagggtgtgaaaaagtcagtgatgctccccctttctactccagatcctgtccttcctggagcaaggttgagggagtaggttttgaagagtcccttaatatgtggtggaacaggccaggagttagagaaagggctggcttctgtttacctgctcactggctctagccagcccagggaccacatcaatgtgagaggaagcctccacctcatgttttcaaacttaatactggagactggctgagaacttacggacaacatcctttctgtctgaaacaaacagtcacaagcacaggaagaggctgggggactagaaagaggccctgccctctagaaagctcagatcttggcttctgttactcatactcgggtgggctccttagtcagatgcctaaaacattttgcctaaagctcgatgggttctggaggacagtgtggcttgtcacaggcctagagtctgagggaggggagtgggagtctcagcaatctcttggtcttggcttcatggcaaccactgctcacccttcaacatgcctggtttaggcagcagcttgggctgggaagaggtggtggcagagtctcaaagctgagatgctgagagagatagctccctgagctgggccatctgacttctacctcccatgtttgctctcccaactcattagctcctgggcagcatcctcctgagccacatgtgcaggtactggaaaacctccatcttggctcccagagctctaggaactcttcatcacaactagatttgcctcttctaagtgtctatgagcttgcaccatatttaataaattgggaatgggtttggggtattaatgcaatgtgtggtggttgtattggagcagggggaattgataaaggagagtggttgctgttaatattatcttatctattgggtggtatgtgaaatattgtacatagacctgatgagttgtgggaccagatgtcatctctggtcagagtttacttgctatatagactgtacttatgtgtgaagtttgcaagcttgctttagggctgagccctggactcccagcagcagcacagttcagcattgtgtggctggttgtttcctggctgtccccagcaagtgtaggagtggtgggcctgaactgggccattgatcagactaaataaattaagcagttaacataactggc

SEQ ID NO: 14 - protein, 638 aa, Homo sapiens
MSALAGEDVWRCPGCGDHIAPSQIWYRTVNETWHGSCFRCSECQDSLTNWYYEKDGKLYCPKDYWGKFGEFCHGCSLLMTGPFMVAGEFKYHPECFACMSCKVIIEDGDAYALVQHATLYCGKCHNEVVLAPMFERLSTESVQEQLPYSVTLISMPATTEGRRGFSVSVESACSNYATTVQVKEVNRMHISPNNRNAIHPGDRILEINGTPVRTLRVEEVEDAISQTSQTLQLLIEHDPVSQRLDQLRLEARLAPHMQNAGHPHALSTLDTKENLEGTLRRRSLRRSNSISKSPGPSSPKEPLLFSRDISRSESLRCSSSYSQQIFRPCDLIHGEVLGKGFFGQAIKVTHKATGKVMVMKELIRCDEETQKTFLTEVKVMRSLDHPNVLKFIGVLYKDKKLNLLTEYIEGGTLKDFLRSMDPFPWQQKVRFAKGIASGMAYLHSMCIIHRDLNSHNCLIKLDKTVVVADFGLSRLIVEERKRAPMEKATTKKRTLRKNDRKKRYTVVGNPYWMAPEMLNGKSYDETVDIFSFGIVLCEIIGQVYADPDCLPRTLDFGLNVKLFWEKFVPTDCPPAFFPLAAICCRLEPESRPAFSKLEDSFEALSLYLGELGIPLPAELEELDHTVSMQYGLTRDSPP

SEQ ID NO: 15 - DNA, 2169 bp, Homo sapiens
ccgcctccgagtgccttgcgcggacctgagctggagatgctggccgggctaccgacgtcagaccccgggcgcctcatcacggacccgcgcagcggccgcacctacctcaaaggccgcttgttgggcaaggggggcttcgcccgctgctacgaggccactgacacagagactggcagcgcctacgctgtcaaagtcatcccgcagagccgcgtcgccaagccgcatcagcgcgagaagatcctaaatgagattgagctgcaccgagacctgcagcaccgccacatcgtgcgtttttcgcaccactttgaggacgctgacaacatctacattttcttggagctctgcagccgaaagtccctggcccacatctggaaggcccggcacaccctgttggagccagaagtgcgctactacctgcggcagatcctttctggcctcaagtacttgcaccagcgcggcatcttgcaccgggacctcaagttgggaaattttttcatcactgagaacatggaactgaaggtgggggattttgggctggcagcccggttggagcctccggagcagaggaagaagaccatctgtggcacccccaactatgtggctccagaagtgctgctgagacagggccacggccctgaagcggatgtatggtcactgggctgtgtcatgtacacgctgctctgcgggagccctccctttgagacggctgacctgaaggagacgtaccgctgcatcaagcaggttcactacacgctgcctgccagcctctcactgcctgcccggcagctcctggccgccatccttcgggcctcaccccgagaccgcccctctattgaccagatcctgcgccatgacttctttaccaagggctacacccccgatcgactccctatcagcagctgcgtgacagtcccagacctgacaccccccaacccagctaggagtctgtttgccaaagttaccaagagcctctttggcagaaagaagaagagtaagaatcatgcccaggagagggatgaggtctccggtttggtgagcggcctcatgcgcacatccgttggccatcaggatgccaggccagaggctccagcagcttctggcccagcccctgtcagcctggtagagacagcacctgaagacagctcaccccgtgggacactggcaagcagtggagatggatttgaagaaggtctgactgtggccacagtagtggagtcagccctttgtgctctgagaaattgtatagctttcatgcccccagcggaacagaacccggcccccctggcccagccagagcctctggtgtgggtcagcaagtgggttgactactccaataagttcggctttgggtatcaactgtccagccgccgtgtggctgtgctcttcaacgatggcacacatatggccctgtcggccaacagaaagactgtgcactacaatcccaccagcacaaagcacttctccttctccgtgggtgctgtgccccgggccctgcagcctcagctgggtatcctgcggtacttcgcctcctacatggagcagcacctcatgaagggtggagatctgcccagtgtggaagaggtagaggtacctgctccgcccttgctgctgcagtgggtcaagacggatcaggctctcctcatgctgtttagtgatggcactgtccaggtgaacttctacggggaccacaccaagctgattctcagtggctgggagcccctccttgtgacttttgtggcccgaaatcgtagtgcttgtacttacctcgcttcccaccttcggcagctgggctgctctccagacctgcggcagcgactccgctatgctctgcgcctgctccgggaccgcagcccagcttaggacccaagccctgaaggcctgaggcctgtgcctgtcaggctctggcccttgcctttgtggccttcccccttcctttggtgcctcactgggggctttgggccgaatcccccagggaatcagggaccagctttactggagttgggggcggcttgtcttcgctggctcctaccccatctccaagataagcctgagccttagctcccagctagggggcgttatttatggaccacttttatttattgtcagacacttatttattgggatgtgagccccagggggcctcctcctaggataataaacaattttgca

SEQ ID NO: 16 - protein, 607 aa, Homo sapiens
MLAGLPTSDPGRLITDPRSGRTYLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFFTKGYTPDRLPISSCVTVPDLTPPNPARSLFAKVTKSLFGRKKKSKNHAQERDEVSGLVSGLMRTSVGHQDARPEAPAASGPAPVSLVETAPEDSSPRGTLASSGDGFEEGLTVATVVESALCALRNCIAFMPPAEQNPAPLAQPEPLVWVSKWVDYSNKFGFGYQLSSRRVAVLFNDGTHMALSANRKTVHYNPTSTKHFSFSVGAVPRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 17 - DNA, 3492 bp, Homo sapiens
cagctaagacccggagaggtggaatttcactttgaaattcccttgcctcgtgagggccggcgctgggcatgctcagtagccgcggcgctgctgctgggctgctgggctggcgcggagtccaccctgccgtctccgccttggcttctgggcgtccagaaggccaggcatttgccgcctctgagcgcttctgttccccttacccgcaacctcctactgctcttcctctctccctctcttagggaggttgaagctggtgctggtttctgtcggcgccacagactgactgctctgcaaaccccagccgaggacctgaatcccggagactagaagacccttggcggtggctctttctaatagcactttacctgaagtggggtcgtggtggagtttctcctccacctctcaatgcaaacactatgcggagagcagtctgcttccctgcgctgtgcctgctccttaatcttcacgctgcagggtgcttttcaggaaacaatgatcattttttggcaattaatcagaagaagagtgggaagccggtattcatttataagcattcacaagacattgagaagagcctggatatagccccacaaaaaatctacagacatagctaccattcctcttccgaagctcaagtaagcaaacgccaccagattgtcaattcagcatttcctagacccgcatatgacccgtctctcaatctgctggccatggatggtcaagatcttgaagtggaaaatctcccaatcccagcagcaaatgtaattgtggtgacactgcaaatggatgtaaacaagctgaacataaccttgcttcggatcttccgccaaggagtggctgcagctttaggactcttaccccagcaagtgcacatcaatcgcctcattggaaagaagaacagtattgaactgtttgtgtctcccataaaccgaaaaacaggaatttctgatgctctgccctctgaggaagttcttcgttcacttaatatcaatgttttgcatcaaagtttatcccagtttggaattacagaagtctctcctgagaaaaatgttttacaagggcagcatgaagcggacaaaatctggagcaaagaaggattttatgctgttgtcatttttctcagcatctttgttattatagtaacgtgtttgatgattctttacagattaaaagaaagatttcagctttccttaagacaagacaaagagaaaaaccaggagatccacctatcgcccatcacattacagccagcactgtccgaggcaaagacagtccacagcatggtccaacctgagcaggccccaaaggtactgaatgttgtcgtggaccctcaaggccgaggtgctcctgagatcagagctaccaccgctacctctgtttgcccttctcctttcaaaatgaagcccataggacttcaagagagaagagggtccaacgtatctcttacattggacatgagtagcttggggaacattgaaccctttgtgtctataccaacaccacgggagaaggtagcaatggagtatctgcagtcagccagccgaattctcacaaggtctcagctgagggacgtcgtggcaagttcacatttactccaaagtgaattcatggaaataccgatgaactttgtggatcccaaagaaattgatattccgcgtcatggaactaaaaatcgctataagaccattttaccaaatcccctcagcagagtgtgtttaagaccaaaaaatgtaaccgattcattgagcacctacattaatgctaattatattaggggctacagtggcaaggagaaagccttcattgccacgcagggccccatgatcaacaccgtggatgatttctggcagatggtttggcaggaagacagccctgtgattgttatgatcacaaaactcaaagaaaaaaatgagaaatgtgtgctatactggccggaaaagagagggatatatggaaaagttgaggttctggttatcagtgtaaatgaatgtgataactacaccattcgaaaccttgtcttaaagcaaggaagccacacccaacatgtgaagcattactggtacacctcatggcctgatcacaagactccagacagtgcccagcccctcctacagctcatgctggatgtagaagaagacagacttgcttcccagggccgagggcctgtggttgtccactgcagtgcaggaataggtagaacagggtgttttattgctacatccattggctgtcaacagctgaaagaagaaggagttgtggatgcactaagcattgtctgccagcttcgtatggatagaggtggaatggtccaaaccagtgagcagtatgaatttgtgcaccatgctctgtgcctgtatgagagcagactttcagcagagactgtccagtgagtcattgaagacttgtcagaccatcaatctcttggggtgattaatcaaattacccacccaaggcttctagaaggagcttcctgcaatggaaggaaggagaagctctgaagcccatgtatggcatggattgtggaagactgggcaacatatttaagatttccagctccttgtgtatatgaatgcatttgtaagcatcccccaaattattctgaaggttttttgatgatggaggtatgataggtttatcacacagcctaaggcagattttgttttgtctgtactgactctatctgccacacagaatgtatgtatgtaatattcagtaataaatgtcatcaggtgatgactggatgagctgctgaagacattcgtattatgtgttagatgctttaatgtttgcaaaatctgccttgtgaatggactgtcagctgttaaactgttcctgttttgaagtgctattacctttctcagttaccagaatcttgctgctaaagttgcaagtgattgataatggatttttaacagagaagtctttgtttttgaaaaacaaaaatcaaaaacagtaactattttatatggaaatgtgtcttgataatattacctattaaatgtgtatttatagtccctcctatcaaacaattacagagcacaatgattgtcattgggtatatatgtatttactctctattattgggcataaaggtggcttctgctccagaactctatccactgtatttccacatcgtgagtcattttactttaaaagggaaaaacaaatttgtagcaactctgaagtatcaagagttttaactacttgactctcttttgctaagaagggatttttgaatatgctatctacctggaatctctctctcaacaaaaggtatatgccttcaggaatgatataatctgtcccattttcgaggctccttataaggacatttccatgtatgtccttacatttctgaaagctttcaatcttcaagagccaaaaaaaattaaaataactaccctcagcaaacactagctgttctgctcatatatgaatttttaatgcagcaatgttgactttgtttcatactgccaataaactcttaatact

SEQ ID NO: 18 - protein, 657 aa, Homo sapiens
MRRAVCFPALCLLLNLHAAGCFSGNNDHFLAINQKKSGKPVFIYKHSQDIEKSLDIAPQKIYRHSYHSSSEAQVSKRHQIVNSAFPRPAYDPSLNLLAMDGQDLEVENLPIPAANVIVVTLQMDVNKLNITLLRIFRQGVAAALGLLPQQVHINRLIGKKNSIELFVSPINRKTGISDALPSEEVLRSLNINVLHQSLSQFGITEVSPEKNVLQGQHEADKIWSKEGFYAVVIFLSIFVIIVTCLMILYRLKERFQLSLRQDKEKNQEIHLSPITLQPALSEAKTVHSMVQPEQAPKVLNVVVDPQGRGAPEIRATTATSVCPSPFKMKPIGLQERRGSNVSLTLDMSSLGNIEPFVSIPTPREKVAMEYLQSASRILTRSQLRDVVASSHLLQSEFMEIPMNFVDPKEIDIPRHGTKNRYKTILPNPLSRVCLRPKNVTDSLSTYINANYIRGYSGKEKAFIATQGPMINTVDDFWQMVWQEDSPVIVMITKLKEKNEKCVLYWPEKRGIYGKVEVLVISVNECDNYTIRNLVLKQGSHTQHVKHYWYTSWPDHKTPDSAQPLLQLMLDVEEDRLASQGRGPVVVHCSAGIGRTGCFIATSIGCQQLKEEGVVDALSIVCQLRMDRGGMVQTSEQYEFVHHALCLYESRLSAETVQ

SEQ ID NO: 19 - DNA, 985 bp, Homo sapiens
ccggcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattc

SEQ ID NO: 20 - protein, 312 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEF

(19) United States
(12) Patent Application Publication
(10) Pub. No.: US 2003/0176443 A1
(43) Pub. Date: Sep. 18, 2003

(54) Pyridylpyrimidine derivatives as effective compounds against prion diseases

(76) Inventors: Matthias Stein-Gerlach, Munich (DE); Konstadinos Salassidis, Ehcing (DE); Gerald Bacher, Germering (DE); Stefan Muller, Munich (DE)

Correspondence Address: Leon R Yankwich, Yankwich & Associates, 201 Broadway, Cambridge, MA, 02139, US

(21) Appl. No.: 10/204,041

(22) PCT Filed: May 16, 2002
(86) PCT No.: PCT/EP02/05420

(30) Foreign Application Priority Data
May 16, 2001 (EP) 01111858.5
Jul. 13, 2001 (EP) 01117113.9

(51) Int. Cl. A61K 31/506; C 07D 4 3/14; C 07D 4 3/02

(52) U.S. Cl. 514/252.18; 514/275; 544/295; 544/331

(57) ABSTRACT

The present invention relates to pyridylpyrimidine derivatives of the general formula (I):

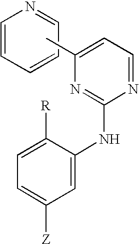

wherein R represents hydrogen or methyl and Z represents nitrogen containing functional groups, the use of the pyridylpyrimidine derivatives as pharmaceutically active agents, especially for the prophylaxis and/or treatment of prion infections and prion diseases, as well as compositions containing at least one pyridylpyrimidine derivative and/or pharmaceutically acceptable salt thereof. Furthermore, the present invention is directed to methods for preventing and/or treating prion infections and prion diseases using said pyridylpyrimidine derivatives. Human cellular protein kinases, phosphatases and cellular signal transduction molecules are disclosed as targets for detecting, preventing and/or treating prion infections and diseases, especially BSE, vCJD, or CJD which can be inhibited by the inventive pyridylpyrimidine derivatives.

The present invention relates to pyridylpyrimidine derivatives, the use of the pyridylpyrimidine derivatives as pharmaceutically active agents, especially for the prophylaxis and/or treatment of prion infections and prion diseases, as well as compositions containing at least one pyridylpyrimidine derivative and/or pharmaceutically acceptable salt thereof, and methods for preventing and/or treating prion infections and prion diseases. Furthermore, human cellular protein kinases, phosphatases and cellular signal transduction molecules are disclosed as targets for detecting, preventing and/or treating prion infections and diseases, especially BSE, vCJD, or CJD.

### BACKGROUND OF THE INVENTION

Pyridylpyrimidine derivatives are known from WO 9509851 as effective compounds for chemotherapy of tumors, from WO 9509853, EP-A-0 588 762, WO 9509847, WO 9903854, and EP-B-0 564 409 as effective compounds for treatment of tumors. Furthermore, EP-B-0 564 409 discloses the use of said compounds in the treatment of artherosclerosis and Exp. Opin. Ther. Patents, 1998, 8(12), 1599-1625 describes the use of pyridylpyrimidine derivatives, especially of Gleevec™, the Novartis compound CGP 57148, as tyrosine kinase inhibitors in cancer treatment.

Prions are infectious agents which do not have a nucleic acid genome. It seems that a protein alone is the infectious agent. A prion has been defined as “small proteinaceous infectious particle which resists inactivation by procedures that modify nucleic acids”. The discovery that proteins alone can transmit an infectious disease has come as a considerable surprise to the scientific community. Prion diseases are often called “transmissible spongiform encephalopathies”, because of the post mortem appearance of the brain with large vacuoles in the cortex and cerebellum. Probably most mammalian species develop these diseases. Prion diseases are a group of neurodegenerative disorders of humans and animals and the prion diseases can manifest as sporadic, genetic or infectious disorders. Examples for prion diseases acquired by exogenous infection are the Bovine spongiform encephalitis (BSE) of cattle and the new variant of Creutzfeld-Jakob disease (vCJD) caused by BSE. Further examples include kuru, Gerstmann-Sträussler-Scheinker disease of humans as well as scrapie of animals. For many years, the prion diseases were thought to be caused by viruses despite intriguing evidence to the contrary. The unique characteristic common to all of these disorders, whether sporadic, dominantly inherited, or acquired by infection, is that they involve the aberrant metabolism of the prion protein (PrP). In many cases, the cellular prion protein (PrP^{c}) [“c” refers to cellular] is converted into the scrapie isoform (PrP^{Sc}) [“Sc” refers to Scrapie] by a posttranslational process that involves a conformational change. Often, the human prion diseases are transmissible to experimental animals and all of the inherited prion diseases segregate with PrP gene mutations.

These prion diseases in animals and humans have a long incubation period and a long clinical course, and are always fatal leading via decerebration to death within an average period of 7 months (CJD). Neuropathological features consist of neuronal vacuolization, neuronal death and gliosis with hyperastrocytosis. The precise diagnosis of transmissible neurodegenerative diseases can be established only by the examination of the central nervous system after biopsy or autopsy.

Clinical symptoms of the disease are progressive dementia, myoclonus and prominent ataxia with the additional clinical features of dysautonomia and delirious psychomotor excitement and with relatively preserved verbal responses.

Between 1980 and, roughly, 1996, about 750,000 cattle infected with BSE were slaughtered for human consumption in Great Britain (Anderson, R. M. et al. Nature 382, 779-788,1996; Ferguson, N. M., Donnelly, C. A., Woolhouse, M. E. J. & Anderson, R. M. Phil. Trans. R. Soc. Lond. B 352, 803-838, 1997). The annual incidence of vCJD (3, 10, 10, 18, 14 and 33 deaths in 1995-2000, respectively) can be interpreted as a first sign of a steady or exponential increase over the next years. The suggestion by the European Union Scientific Steering Committee that up to 500,000 people could have been exposed to BSE from a single infected bovine has fuelled speculation that millions of consumers are at risk.

Recent findings demonstrate that the pathogenic PrP^{Sc} of vCJD can be found in the lymph system (e.g. tonsils, lymph nodes) in humans suggesting a high risk of horizontal spread via lymph and/or blood transmission, dramatically increasing the number of people at risk.

The medical need in prion diseases today can be clearly defined as the establishment of a diagnostic system, that can detect the disease as early as possible in living humans and/or animals, to estimate the medical need for the treatment in the future and to identify the infected animals to remove them from the food chain. The medical need for prion diseases in the future (approximately starting in 5-10 years) will be medical treatment that inhibits the disease symptoms, the manifestation and/or progression of the disease.

It is object of the present invention to provide novel and also known compounds which can be used as pharmaceutically active agents, especially for prophylaxis and/or treatment of prion infections and prion diseases, methods wherein said compounds are used in order to treat prion infections and prion diseases and compositions containing at least one inventive compound and/or pharmaceutically acceptable salt thereof as a pharmaceutically active ingredient.

The object of the present invention is solved by the teaching of the independent claims. Further advantageous features, aspects and details of the invention are evident from the dependent claims, the description, the examples, and the figures of the present application.

### DESCRIPTION OF THE INVENTION

One aspect of the present invention is related to compounds of the general formula (I):

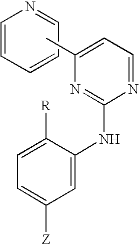

wherein:

R represents hydrogen or methyl;

Y, Y′, Y″ are independently of each other —H, —F, —Cl, —Br, —I, —CH₂F, —CH₂Cl, —CH₂Br, —CH₂I, —OH, —OCH₃, —CH₃, —CN, —OCF₃, 4-methylpiperazin-1-yl-methyl, —C(CH₃)═N—NH—C(NH)—NH₂;

Z represents —NO₂, —NH₂, —NH—CO—X, —NH—CS—X, —NH—CO—NH—X, —NH—SO₂—X;

X represents thiophenyl, cyclohexyl, isoquinolinyl, naphthyl, quinolinyl, cyclopentyl, pyridinyl, naphthyridinyl, or

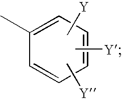

and pharmaceutically acceptable salts thereof.

Another aspect of the present invention relates to the use of compounds of the general formula (I):

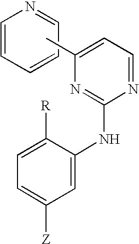

wherein:

R represents hydrogen or methyl;

Y, Y′, Y″ are independently of each other —H, —F, —Cl, —Br, —I, —CH₂F, —CH₂Cl, —CH₂Br, —CH₂I, —OH, —OCH₃, —CH₃, —CN, —OCF₃, 4-methylpiperazin-1-yl-methyl, —C(CH₃)═N—NH—C(NH)—NH₂;

Z represents —NO₂, —NH₂, —NH—CO—X, —NH—CS—X, —NH—CO—NH—X, —NH—SO₂—X;

X represents thiophenyl, cyclohexyl, isoquinolinyl, naphthyl, quinolinyl, cyclopentyl, pyridinyl, naphthydinyl, or

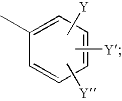

and pharmaceutically acceptable salts thereof as pharmaceutically active agents, especially for prophylaxis and/or treatment of infectious diseases, or in a more general sense, for prophylaxis and/or treatment of nerodegenerative diseases.

Thus, one embodiment of the present invention disclosed herein is directed to a method for preventing and/or treating infections and/or diseases associated with said infections in an individual. Said method comprises administering to the individual an amount of at least one compound according to general formula (I) and/or pharmaceutically acceptable salts thereof effective to prevent and/or treat said infections and/or diseases. Most preferred is the administration of a compound 53.

As revealed for the first time herein, the present invention discloses the use of compounds of the general formula (I) for the prophylaxis and/or treatment of prion infections and prion diseases. As described above, said pyridylpyrimidine derivatives have first of all been used in tumor therapy. The Novartis compound Gleevec™ also known as Glivec™, CGP-57148B, imatinib mesylate, STI-571, STI-571A, CAS 152459-95-5, or 4-((Methyl-1-piperazinyl)methyl)-N-[4-methyl-3-[[4-(3-pyridinyl)-2-pyrimidinyl]amino]-phenyl]benzamide methanesulfonate, has been registered in many countries as anticancer drug. This Gleevec™ compound (compound 53) is also the most active one in the indication prion diseases.

The name “prion” is used to describe the causative agents which underlie the transmissible spongiform encephalopathies. A prion is proposed to be a novel infectious particle that differs from viruses and viroids. It is composed solely of one unique protein that resists most inactivation procedures such as heat, radiation, and proteases. The latter characteristic has led to the term protease-resistant isoform of the prion protein. The protease-resistant isoform has been proposed to slowly catalyze the conversion of the normal prion protein into the abnormal form.

The term “isoform” in the context of prions means two proteins with exactly the same amino acid sequence that are folded into molecules with dramatically different tertiary structures. The normal cellular isoform of the prion protein (PrP^{c}) has a high α-helix content, a low β-sheet content, and is sensitive to protease digestion. The abnormal, disease-causing isoform (PrP^{Sc}) has a lower α-helix content, a much higher β-sheet content, and is much more resistant to protease digestion.

Moreover, in a more general sense, the present invention is concerned with the prophylaxis ans/or treatment of neurodegenerative diseases. For example, Alzheimer is a well-known neurodegenerative disease.

Preferred are the compounds wherein R represents hydrogen. Also preferred are compounds wherein Z represents —NH—CO—X or —NH—SO₂—X and/or wherein Y, Y′, Y″ are independently of each other —H, —F, —Cl, CH₂F, —CH₂Cl, —OH, —OCH₃, —CN, —OCF₃, or a 4-methylpiperazin-1-yl-methyl residue.

Also preferred are the following pyridylpyrimidine derivatives selected from the group comprising:

Compound 1: (3-Nitrophenyl)4-pyridin-3-yl-pyrimidin-2-yl)-amine;

Compound 2: (3-Aminophenyl)-(4-pyridin-3-yl-pyrimidin-2-yl)-amine;

Compound 3: (5-Amino-2-methylphenyl)-(4-pyridin-3-yl-pyrimidin-2-yl)-amine;

Compound 4: 4-Chloromethyl-N-[4-methyl-3-(4-pyridin-3-yl-pyrimidin-2-ylamino)-phenyl]-benzamide;

Compound 5: 4-Chloromethyl-N-[3-(4-pyridin-3-yl-pyrimidin-2-ylamino)-phenyl]-benzamide;

Compound 6: 4-(4-Methylpiperazin-1-ylmethyl)-N-[3-(4-pyridin-3-yl-pyrimidin-2-ylamino)-phenyl]-benzamide;

Compound 7: Thiophene-3-carboxylic acid [4-methyl-3-(4-pyridin-4-yl-pyrimidin-2-ylamino)-pheny]-amide;

Compound 8: 4-Chloro-N-[4-methyl-3-(4-pyridin-3-yl-pyrimidin-2-ylamino)-phenyl]-benzamide;

Compound 9: 4-Chloro-N-[3-(4-pyridin-3-yl-pyrimidin-2-ylamino)-phenyl]-benzamide;

Compound 10: 3,4,5-Trimethoxy-N-[3-(4-pyridin-3-yl-pyrimidin-2-ylamino)-phenyl]-benzamide;

Compound 11: 4-Cyano-N-[3-(4-pyridin-3-yl-pyrimidin-2-ylamino)-phenyl]-benzamide;

Compound 12: 4-Methoxy-N-[3-(4-pyridin-3-yl-pyrimidin-2-ylamino)-phenyl]-benzamide;

Compound 13: 4-Chloro-N-[3-(4-pyridin-3-yl-pyrimidin-2-ylamino)-phenyl]-benzenesulfonamide;

Compound 14: Thiophene-3-carboxylic acid [3-(4-pyridin-3-yl-pyrimidin-2-ylamino)-phenyl]-amide;

Compound 15: 3,5-Dimethoxy-N-[3-(4-pyridin-3-yl-pyrimidin-2-ylamino)-phenyl]-benzamide;

Compound 16: 3,4,5-Trimethoxy-N-[4-methyl-3-(4-pyridin-3-yl-pyrimidin-2-ylamino)-phenyl]-benzamide;

Compound 17: 4-Cyano-N-[4-methyl-3-(4-pyridin-3-yl-pyrimidin-2-ylamino)-phenyl]-benzamide;

Compound 18: 4-Methoxy-N-[4-methyl-3-(4-pyridin-3-yl-pyrimidin-2-ylamino)-phenyl]-benzamide;

Compound 19: 4-Chloro-N-[4-methyl-3-(4-pyridin-3-yl-pydmidin-2-ylamino)-phenyl]-benzenesulfonamide;

Compound 20: Thiophene-3-carboxylic acid [4-methyl-3-(4-pyridin-3-yl-pyrimidin-2-ylamino)-phenyl]-amide;

Compound 21: 3,5-Dimethoxy-N-[4-methyl-3-(4-pyridin-3-yl-pyrimidin-2-ylamino)-phenyl]-benzamide;

Compound 22: 4-Trifluoromethoxy-N-[4-methyl-3-(4-pyridin-3-yl-pyrimidin-2-ylamino)-phenyl]-benzamide;

Compound 23: Cyclohexanecarboxylic acid [4-methyl-3-(4-pyridin-3-yl-pyrimidin-2-ylamino)-phenyl]-amide;

Compound 24: Cyclohexanecarboxylic acid [3-(4-pyridin-3-yl-pyrimidin-2-ylamino)-phenyl]-amide;

Compound 25: Isoquinoline-5-sulfonic acid [4-methyl-3-(4-pyridin-3-yl-pyrimidin-2-ylamino)-phenyl]-amide;

Compound 26: Isoquinoline-5-sulfonic acid [3-(4-pyridin-3-yl-pyrimidin-2-ylamino)-phenyl]-amide;

Compound 27: (5-Nitro-2-methylphenyl)-(4-pyridin-2-yl-pyrimidin-2-yl)-amine;

Compound 28: (5-Amino-2-methylphenyl)-(4-pyridin-2-yl-pyrimidin-2-yl)-amine;

Compound 29: 3,4,5-Trimethoxy-N-[4-methyl-3-(4-pyridin-2-yl-pyrimidin-2-ylamino)-phenyl]-benzamide;

Compound 30: 4-Cyano-N-[4-methyl-3-(4-pyridin-2-yl-pyrimidin-2-ylamino)-phenyl]-benzamide;

Compound 31: (3-Aminophenyl)-(4-pyridin-2-yl-pyrimidin-2-yl)-amine;

Compound 32: 4-Chloro-N-[4-methyl-3-(4-pyridin-2-yl-pyrimidin-2-ylamino)-phenyl]-benzamide;

Compound 33: Cyclohexanecarboxylic acid [4-methyl-3-(4-pyridin-2-yl-pyrimidin-2-ylamino)-phenyl]-amide;

Compound 34: 4-Cyano-N-[4-methyl-3-(4-pyridin-4-yl-pyrimidin-2-ylamino)-phenyl]-benzamide;

Compound 35: 4-Chloro-N-[4-methyl-3-(4-pyridin-4-yl-pyrimidin-2-ylamino)-phenyl]-benzenesulfonamide;

Compound 36: 4-Methoxy-N-[4-methyl-3-(4-pyridin4-yl-pyrimidin-2-ylamino)-phenyl]-benzamide;

Compound 37: 4-Chloro-N-[4-methyl-3-(4-pyridin4-yl-pyrimidin-2-ylamino)-phenyl]-benzamide;

Compound 38: Cyclohexanecarboxylic acid [3-(4-pyridin-4-yl-pyrimidin-2-ylamino)-phenyl]-amide;

Compound 39: 3,5-Dimethoxy-N-[3-(4-pyridin-4-yl-pyrimidin-2-ylamino)-phenyl]-benzamide;

Compound 40: (5-Amino-2-methylphenyl)-(4-pyridin-4-yl-pyrimidin-2-yl)-amine;

Compound 41: Thiophene-3-carboxylic acid [3-(4-pyridin-4-yl-pyrimidin-2-ylamino)-phenyl]-amide;

Compound 42: 4-Chloro-N-[3-(4-pyridin-4-yl-pyrimidin-2-ylamino)-phenyl]-benzenesulfonamide;

Compound 43: 4-Chloro-N-[3-(4-pyridin-4-yl-pyrimidin-2-ylamino)-phenyl]-benzamide;

Compound 44: (3-Aminophenyl)-(4-pyridin-4-yl-pyrimidin-2-yl)-amine;

Compound 45: (3-Nitrophenyl)-(4-pyridin-4-yl-pyrimidin-2-yl)-amine;

Compound 46: 4-Trifluoromethoxy-N-[4-methyl-3-(4-pyridin-4-yl-pyrimidin-2-ylamino)-phenyl]-benzamide;

Compound 47: Isoquinoline-5-sulfonic acid [3-(4-pyridin-4-yl-pyrimidin-2-ylamino)-phenyl]-amide;

Compound 48: 4-Methoxy-N-[3-(4-pyridin-4-yl-pyrimidin-2-ylamino)-phenyl]-benzamide;

Compound 49: 4Cyano-N-[3-(4-pyridin-4-yl-pyrimidin-2-ylamino)-phenyl]-benzamide;

Compound 50: 3,4,5-Trimethoxy-N-[3-(4-pyridin-4-yl-pyrimidin-2-ylamino)-phenyl]-benzamide;

Compound 51: 3,5-Dimethoxy-N-[4-methyl-3-(4-pyridin-4-yl-pyrimidin-2-ylamino)-phenyl]-benzamide;

Compound 52: 3,4,5-Trimethoxy-N-[4-methyl-3-(4-pyridin-4-yl-pyrimidin-2-ylamino)-phenyl]-benzamide;

Compound 53: 4-(4-Methylpiperazin-1-ylmethyl)-N-[4-methyl-3-(4-pyridin-3-yl-pyrimidin-2-ylamino)-phenyl]-benzamide (Gleevec™);

Compound 54: 4-Methyl-N-[4-methyl-3-(4-pyridin-2-yl-pyrimidin-2-ylamino)-phenyl]-benzenesulfonamide

Compound 55: 4-Methoxy-N-[4-methyl-3-(4-pyridin-2-yl-pyrimidin-2-ylamino)-phenyl]-benzamide;

Compound 56: 3,5-Dimethoxy-N-[4-methyl-3-(4-pyridin-2-yl-pyrimidin-2-ylamino)-phenyl]-benzamide;

Compound 57: Naphthalene-2-carboxylic acid [4-methyl-3-(4-pyridin-3-yl-pyrimidin-2-ylamino)-phenyl]-amide;

Compound 58: N-[3-(4-Pyridin-4-yl-pyrimidin-2-ylamino)-phenyl]-benzamide;

Compound 59: 4-Chloro-N-[3-(4-pyridin-2-yl-pyrimidin-2-ylamino)-phenyl]-benzamide;

Compound 60: 4-Methoxy-N-[3-(4-pyridin-2-yl-pyrimidin-2-ylamino)-phenyl]-benzamide;

Compound 61: 4-Chloro-N-[3-(4-pyridin-2-yl-pyrimidin-2-ylamino)-phenyl]-benzenesulfonamide;

Compound 62: Thiophene-2-carboxylic acid 3-(4-pyridin-2-yl-pyrimidin-2-yl-amino)-phenyl]-amide;

Compound 63: Naphthalene-2-sulfonic-acid [3-(4-pyridin-2-yl-pydimidin-2-yl-amino)-phenyl]-amide;

Compound 64: Isoquinoline-5-sulfonic-acid [3-(4-pyridin-2-yl-pyrimidin-2-yl-amino)-phenyl]-amide;

Compound 65: Cylopentanecarboxylic acid 3-(4-pyridin-2-yl-pyrimidin-2-yl-amino)-phenyl]-amide;

Compound 66: Naphthalene-2carboxylic acid [3-(4-pyridin-2-yl-pyrimidin-2-ylamino)-phenyl]-amide;

Compound 67: 4-Cyano-N-[3-(4-pyridin-2-yl-pyrimidin-2-ylamino)-phenyl]-benzamide;

Compound 68: 3,5-Dimethoxy-N-[3-(4-pyridin-2-yl-pyrimidin-2-ylamino)-phenyl]-benzamide;

Compound 69: 4-Bromo-N-[3-(4-pyridin-2-yl-pyrimidin-2-ylamino)-phenyl]-benzamide;

Compound 70: 4-Methyl-N-[3-(4-pyridin-2-yl-pyrimidin-2-ylamino)-phenyl]-benzamide;

Compound 71: 4-Fluoro-N-3-(4-pyridin-2-yl-pyrimidin-2-ylamino)-phenyl]-benzenesulfonamide;

Compound 72: 3,5-Dichloro-N-[3-(4-pyridin-2-yl-pyrimidin-2-ylamino)-phenyl]-benzamide;

Compound 73: N-[3-(4-Pyridin-2-yl-pyrimidin-2-ylamino)-phenyl]-benzamide;

Compound 74: 4-Chloromethyl-N-[3-(4-pyridin-2-yl-pyrimidin-2-ylamino)-phenyl]-benzamide;

Compound 75: 4-Methyl-N-3-(4-pyridin-2-yl-pyrimidin-2-ylamino)-phenyl]-benzenesulfonamide

Compound 76: 4-(4-Methylpiperazin-1-ylmethyl)-N-[3-(4-pyridin-2-yl-pyrimidin-2-ylamino)-phenyl]-benzamide;

Compound 77: Naphthalene-2-carboxylic acid [3-(4-pyridin-4-yl-pyrimidin-2-ylamino)-phenyl]-amide;

Compound 78: 2-Methoxy-N-[4-methyl-3-(4-pyridin-3-yl-pyrimidin-2-ylamino)-phenyl]-benzamide;

Compound 79: 2-Methoxy-N-[3-(4-pyridin-3-yl-pyrimidin-2-ylamino)-phenyl]-benzamide;

Compound 80: 4-Methyl-N-[3-(4-pyridin-3-yl-pyrimidin-2-ylamino)-phenyl]-benzamide;

Compound 81: 4-Methyl-N-[4-methyl-3-(4-pyridin-3-yl-pyrimidin-2-ylamino)-phenyl]-benzamide;

Compound 82: N-[4-Methyl-3-(4-pyridin-3-yl-pyrimidin-2-ylamino)-phenyl]-benzamide;

Compound 83: 1(3,5-Diacetyl-phenyl)-3-[4-methyl-3-(4-pyridin-3-yl-pyrimidin-2-ylamino)-pheny]-urea;

Compound 84: 1-{3,5-Bis-(amidinohydrazone)-phenyl}-3-[4-methyl-3-(4-pyridin-3-yl-pyrimidin-2-ylamino)-phenyl]-urea;

Compound 85: N-[4-Methyl-3-(4-pyridin-3-yl-pyrimidin-2-ylamino)-phenyl]-nicotinamide;

Compound 86: N-[3-(4-Pyridin-3-yl-pyrimidin-2-ylamino)-phenyl]-nicotinamide;

Compound 87: [1,8]Naphthyridine-2carboxylic acid [3-(4-pyridin-3-yl-pyrimidin-2-ylamino)-phenyl]-amide;

Compound 88: [1,8]Naphthyridine-2carbothioic acid [3-(4-pyridin-3yl-pyrimidin-2-ylamino)-phenyl]-amide;

Compound 89: 2-Methoxy-N-[3-(4-pyridin-4-yl-pyrimidin-2-ylamino)-phenyl]-benzamide;

Compound 90: 4-Trifluoromethoxy-N-[3-(4-pyridin-4-yl-pyrimidin-2-ylamino)-phenyl]-benzamide;

Compound 91: 4-Methyl-N-[3-(4-pyridin-4-yl-pyrimidin-2-ylamino)-phenyl]-benzamide;

and pharmaceutically active salts of these compounds.

Recent research has revealed how cells communicate with each other to coordinate the growth and maintenance of the multitude of tissues within the human body. A key element of this communication network is the transmission of a signal from the exterior of a cell to its nucleus, which results in the activation or suppression of specific genes. This process is called signal transduction.

An integral part of signal transduction is the interaction of ligands, their receptors and intracellular signal transduction molecules. Ligands are messengers that bind to specific receptors on the surface of target cells. As a result of the binding, the receptors trigger the activation of a cascade of downstream signaling molecules, thereby transmitting the message from the exterior of the cell to its nucleus. When the message reaches the nucleus, it initiates the modulation of specific genes, resulting in the production of RNA and finally proteins that carry out a specific biological function. Disturbed activity of signal transduction molecules may lead to the malfunctioning of cells and disease processes. Specifically, interference of the pathogenic PrP^{Sc} from prion diseases with neuronal cells is necessary for the prion protein to induce its neuropathological features such as neuronal vacuolization, neuronal death and gliosis with hyperastrocytosis.

A key element of this communication network is the transmission of a signal from the exterior of a cell to its nucleus, which results in the activation or suppression of specific genes. The human cellular protein kinases Abl and clk1 are two of the enzymes involved in said signal transduction process. As revealed herein said kinases Abl and clk1 serve as targets and are inhibited by the pyridylpyrimidine compounds of the general formula (I). It could be proved that prion infections and/or prion diseases can be treated and also be prevented by the inhibition of said kinase Abl using the inventive pyridylpyrimidine derivatives. Inhibition of the kinase clk1 by said pyridylpyrimidine compounds can be used for the treatment of infections and diseases.

A microarray platform technology consisting of more than 1100 signal transduction cDNAs has been established. The technology is used for the identification of changes in RNA expression patterns as a result of the manipulation of the host cell by PrP^{Sc}. In addition, differential display techniques were used in order to pinpoint these changes to those enzymes which could be potential targets for drug intervention.

Employing this predefined set of signal transduction relevant cDNAs on the filters, the expression pattern of signal transduction mRNAs in neuronal mouse cells transfected with the pathogenic form of the prion protein (PrP^{Sc}) were compared with the same cells transfected with the non-pathogenic wild-type form (PrP^{c}) as a control. Interference of the PrP^{Sc} with the cellular signaling events is reflected in different gene expression when compared to the control cellular situation (PrP^{c}).

Using this technology, the human cellular protein kinases FGF-R1 (also known as fIg, FI-1, FIt-2, or b-FGFR), Tkt (also known as CCK-2, DDR-2, or EDDR, EC Number 2.7.1.112), Abl (also known as c-abl), clk1, MKK7 (also known as SKK4, SAPKK4, SAPKK5, or JNKK2), LIMK-2, CaM-KI, JNK2 (also known as SAPK1a, SAPKalpha), CDC2 (also known as CDK1), PRK, the human cellular protein phosphatases PTP-SL (also known as MCP83), PTP-zeta, the cellular signal transduction molecules HSP86, and GPIR-1 were identified as potential anti-prion disease targets. Said cellular protein kinases, phosphatases and signal transduction molecules are found to be specifically up- or downregulated by PrP^{Sc} in relevant mouse neuronal cells.

Surprisingly, it was found that the following human cellular targets are significantly up- or downregulated in prion infected cells:

| target | regulation |
|---|---|
| FGF-R1 | 3.6 fold stronger |
| Abl | 5.6 fold stronger |
| MKK7 | 4.1 fold stronger |
| CDC2 | 2.0 fold weaker |
| Tkt | 2.1 fold stronger |
| LIMK-2 | 2.1 fold stronger |
| CaM-KI | 2.1 fold stronger |
| JNK2 | 2.0 fold weaker |
| PRK | 2.0 fold weaker |
| PTPzeta | 4.6 fold weaker |
| PTP-SL | 5.0 fold weaker |
| HSP86 | 4.1 fold weaker |
| GPIR-1 | 2.3 fold weaker |

Thus, one aspect of the present invention relates to a method for preventing and/or treating prion infections and/or diseases associated with said prion infections in an individual which comprises administering to the individual an amount of at least one compound of the general formula (I) and/or pharmaceutically acceptable salts thereof effective to prevent and/or treat said prion infections and/or prion diseases. Most preferred is the administration of a compound according to claim 8.

It could be proven that inhibition of one target selected from FGF-R1, Tkt, Abl, clk1, MKK7, LIMK-2, CaM-KI, JNK2, CDC2, PRK, PTP-SL, PTP-zeta, HSP86, and GPIR-1 was effective to treat prion diseases. Therefore, another aspect of the invention relates to a method for preventing and/or treating prion infections and/or prion diseases in an individual comprising the step of administering a pharmaceutically effective amount of at least one compound according of the general formula (I) and/or pharmaceutically acceptable salts thereof which inhibits at least partially the activity of one target selectef from FGF-R1, Tkt, Abl, clk1, MKK7, LIMK-2, CaM-KI, JNK2, CDC2, PRK, PTP-SL, PTP-zeta, HSP86, and GPIR-1.

The nucleoside sequences of the genes coding for the human cellular protein kinase Abl and the protein kinase clk1 and their amino acid sequences are disclosed in form of a sequence listing shown below. The nucleoside and amino acid sequences for the kinase Abl (Accession Number: M14752) and for the kinase clk1 (Accession Numbers: XM002520, NM004071, L29222, L29219) were obtained from NCBI (National Library of Medicine: PubMed).

The compounds of general formula (I) were identified as inhibitors of at least one target selected from the group comprising FGF-R1, Tkt, Abl, clk1, MKK7, LIMK-2, CaM-KI, JNK2, CDC2, PRK, PTP-SL, PTP-zeta, HSP86, GPIR-1 by the use of a method for detecting compounds useful for the prophylaxis and/or treatment of prion infections and/or diseases. Said method comprises

a) contacting a test compound with at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1; and

b) detecting the activity of said human cellular protein kinase, phosphatase or cellular signal transduction molecule.

The activity of a human cellular protein kinase, phosphatase or cellular signal transduction molecule was preferably measured by means of an enzymatic assay.

As used herein, the term “inhibitor” refers to any compound capable of downregulating, decreasing, suppressing or otherwise regulating the amount and/or activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1. Generally, said inhibitors, including suicide inhibitors, may be proteins, oligo- and polypeptides, nucleic acids, genes, small chemical molecules, or other chemical moieties.

The present disclosure teaches for the first time the up- or downregulation of the above-mentioned human cellular protein kinases, phosphatases, or cellular signal transduction molecules specifically involved in prion infections and/or diseases. Thus, the present invention is also directed to a method for detecting prion infections and/or diseases in an individual comprising:

a) providing a sample from said individual; and

b) adding to said sample a pharmaceutically effective amount of at least one pharmaceutically active agent; and

c) detecting activity in said sample of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

As used herein the term “sample” refers to any sample that can be taken from a living animal or human for diagnostic purposes, especially said sample comprises blood, milk, saliva, sputum, excrement, urine, spinal cord liquid, liquor, lachrymal gland liquid, biopsies and all other samples that can be taken from a living animal or human for diagnostic purposes.

The term “individual” preferably refers to mammals, especially humans or ruminants. Ruminants are, for instance, muledeer, elk, cow, cattle, sheep, goat, deer, or buffalo. Minks are an example for mammals which do not belong to the species of ruminants.

As used herein the term “ruminants” refers to an animal, for instance, cattle, sheep, goat, deer, elk, or buffalo that has four separate stomach chambers, and is therefore able to digest a wide range of organic and plant foods. The term “ruminants” refers also to exotic ruminants, like captive nyala, gemsbok, Arabian oryx, eland, kudu, scimitar-horned oryx, ankole, or bison which are also accessible to develop spongiform encephalopathy.

A similar aspect of the present invention is directed to a method for detecting prion infections and/or prion diseases in cells, cell cultures and/or cell lysates comprising:

a) providing said cells, cell cultures and/or cell lysates; and

b) adding to said cells, cell cultures and/or cell lysates a pharmaceutically effective amount of at least one pharmaceutically active agent; and

c) detecting activity in said sample of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

Furthermore, it has been shown that the inhibition of at least one target selected from the group comprising FGF-R1, Tkt, Abl, clk1, MKK7, LIMK-2, CaM-KI, JNK2, CDC2, PRK, PTP-SL, PTP-zeta, HSP86, GPIR-1 has an effect on the production of prions. Therefore, another aspect of the invention relates to a method for regulating the production of prions in an individual or in cells comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

The inventive compounds according to general formula (I) are examples for the above-mentioned pharmaceutically active agent. Preferably the targets FGF-R1, Tkt, Abl, clk1, MKK7, LIMK-2, CaM-KI, and CDC 2 are used with said methods.

Another type of pharmaceutically active agents useful within the methods disclosed herein are monoclonal or polyclonal antibodies which bind to a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1. Thus, a further aspect of the present invention is related to said monoclonal or polyclonal antibodies which bind to a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

Another embodiment of the present invention utilizes the scientific findings that some targets such as JNK2, PRK, PTP-SL, PTP-zeta, HSP86, and GPIR-1 are downregulated during prion infection and that upregulation of the effected target by means of an activator leads to an alternative way of treating prion infections and diseases associated with prion infection.

Thus, a method was developed for regulating the production of prions either in an individual or in cells. Said methods comprise the step of administering an individual or the cells a pharmaceutically effective amount of at least one pharmaceutically active agent wherein said agent activates at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1, or wherein said agent at least partially activates or stimulates the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 .

Preferably the targets JNK2, PRK, PTP-SL, PTP-zeta, HSP86, and GPIR-1 are used within the above-described methods.

Because of the fact that the organism may upregulate a given target such as FGF-R1, Tkt, Abl, clk1, MKK7, LIMK-2, CaM-KI, and CDC 2 in order to compete with the prion infection, it is also a reasonable approach to further support said upregulation by means of an activator. Therefore, the above-mentioned methods apply either to targets which are downregulated but also to targets which are upregulated.

The novel and partially known pyridylpyrimidine compounds of the general formula (I) represent a new class of pharmaceuticals highly useful for the prophylaxis and treatment of prion infections and prion diseases.

Thus, a further aspect of the present invention describes the use of a compound of the general formula (I) and/or pharmaceutically acceptable salts thereof for the manufacture of a pharmaceutical formulation for prophylaxis and/or treatment of prion infections and/or diseases induced or caused by prion infection.

As used herein the Term “prion diseases” refers to transmissible spongiform encephalopathies. This group of neurologic diseases affects humans and many species of animals causing a “sponge-like” degeneration of brain tissue. Among other unique features, all of these diseases are associated with the accumulation of an abnormal form of the prion protein in nerve cells that eventually leads to the death of the host. While prion diseases can all be transmitted from one host to another, it remains contentious as to whether a virus-like infectious agent or the abnormal prion protein itself, the prion, causes the conversion of normal to abnormal protein.

Probably most mammalian species develop prion diseases. Specific examples for animals include:

| | |
|---|---|
| Scrapie | sheep, goat |
| TME | (transmissible mink encephalopathy): mink |
| CWD | (chronic wasting disease): muledeer, deer, elk |
| BSE | (bovine spongiform encephalopathy): cows, cattles |

Humans are also susceptible to several prion diseases. Examples are:

| | |
|---|---|
| CJD | Creutzfeld-Jacob Disease |
| GSS | Gerstmann-Sträussler-Scheinker syndrome |
| FFI | Fatal familial Insomnia |
| Kuru | |
| Alpers Syndrome | |

The human prion diseases include kuru, sporadic Creutzfeldt-Jakob disease (sCJD), familial CJD (fCJD), iatrogenic CJD (iCJD), Gerstmann-Sträussler-Scheinker (GSS) disease, fatal familial insomnia (FFI), and, more recently, new variant CJD (nvCJD or vCJD). In addition to these human diseases, prion-related diseases, have been recognized in several animal hosts. Scrapie is a naturally occurring disease of sheep and goats that causes ataxia, behavioral changes, and a severe pruritus that leads to scraping behavior, from which the disease was named. Additional prion diseases in animals include transmissible mink encephalopathy (TME), chronic wasting disease (CWD) of deer and elk, feline spongiform encephalopathy (FSE), and bovine spongiform encephalopathy (BSE), among others.

The transmissible nature of prion disease was first demonstrated experimentally in 1936 when Cuillé and Chelle transmitted scrapie to a healthy goat by the intraocular administration of scrapie-infected spinal cord. Thirty years later, sCJD was transmitted to chimpanzees. The pathologic feature common to all these diseases is a prominent vacuolation of the gray matter of the brain that produces a “sponge-like” appearance on light microscopy. This histopathologic appearance, coupled with the transmissible nature of these diseases, led to their collective designation as “transmissible spongiform encephalopathies” or TSEs.

The etiologic agent of the TSEs was proposed to be a “slow virus” to explain its transmissible nature and the prolonged incubation period observed during experimental transmission studies. Early experiments suggested that protein may be a critical component of the infectious agent. These studies established the basis for a new form of a transmissible pathogen, one that is composed ostensibly of only protein and lacks any replicative elements such as nucleic acid.

The term “prion” was coined to indicate an infectious agent with proteinlike properties. The unusual properties of the pathogen were demonstrated in early experiments in which conditions that degrade nucleic acids, such as exposure to ionizing and ultraviolet radiation, did not reduce the infectivity of scrapie fractions. On the other hand, treatments that degrade protein, such as prolonged exposure to proteases, correlated with a reduction in infectivity. A protein with relative resistance to protease digestion was found to be consistently present in the brains of animals and humans with TSE. Surprisingly, this protein was found to be one that is normally encoded by a chromosomal gene of the host.

Thus, the question raised, how a normally expressed protein could also be a transmissible pathogen? It was hypothesized and later demonstrated that PrP exists in two major isoforms: the nonpathogenic or cellular form, designated PrP^{c}, and the pathogenic or scrapie-inducing form, designated PrP^{Sc}. Both PrP^{c} and PrP^{Sc} have the same amino acid sequence, yet they differ in their biochemical properties: PrP^{c} is soluble in nondenaturing detergents and completely degraded by proteases, whereas PrP^{Sc} is insoluble in nondenaturing detergents and shows a relative resistance to proteases. Structural studies of PrP^{c} and PrP^{Sc} indicate a difference in the conformation of the two isoforms: PrP^{c} is predominantly helical, whereas PrP^{Sc} contains at least 40% pleated sheet structure. Conversion to this sheet structure appears to be the fundamental event in prion disease. The ultimate mechanism of how cells die coincident with the generation of prions is still unclear. Simple accumulation of pathogenic protein may not be sufficient to explain disease, however, it may constitute a critical step in cellular dysfunction.

It was shown that the pyridylpyrimidine compounds of the general formula (I) are highly effective for the prophylaxis and/or treatment of prion infections and/or prion diseases selected from the group comprising Scrapie, TME, CWD, BSE, CJD, vCJD, GSS, FFI, Kuru, and Alpers Syndrome. Preferably, the pyridylpyrimidine derivatives are used for preventing and/or treating BSE, vCJD, or CJD.

The above-mentioned prion infections and/or diseases associated with prion infections can be treated using the inventive pyridylpyrimidine derivatives by targeting at least one of the human cellular protein kinases, phosphatases or cellular signal transduction molecules selected from the group comprising FGF-R1, Tkt, Abl, clk1, MKK7, LIMK-2, CaM-KI, JNK2, CDC2, PRK, PTP-SL, PTP-zeta, HSP86, GPIR-1. Thereby, the compounds according to general formula (I) act as inhibitors for at least one of the above-mentioned targets and especially as inhibitors for at least one enzyme selected from the group comprising FGF-R1, Tkt, Abl, clk1, MKK7, LIMK-2, CaM-KI, and CDC 2.

According to these findings a further aspect of the present invention is directed to a method for preventing and/or treating prion infections and/or prion diseases in an individual comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

Another aspect is related to a method for preventing and/or treating prion infections and/or prion diseases in cells or cell cultures comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

The inventive pyridylpyrimidine compounds of formula (I) are examples for the above-mentioned inhibitor. Said pyridylpyrimidine compounds and/or pharmaceutically acceptable salts thereof are administered in a dosage corresponding to an effective concentration in the range of 0.01-50 μM, preferably in the range of 0.01-10 μM, more preferably in the range of 0.01-1 μM, and most preferably in the range of 0.01-0.1 μM.

Because of the fact that the targets JNK2, PRK, PTP-SL, PTP-zeta, HSP86, and GPIR-1 are downregulated in cells infected with prions, an upregulation of said targets represents another strategy in order to treat prion infections and diseases like CJD (nvCJD or vCJD) associated with prion infections. Said upregulation can be performed by activators.

An agent that is able to upregulate, increase, activate, or stimulate the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1, but especially of JNK2, PRK, PTP-SL, PTP-zeta, HSP86, and GPIR-1 is named “activator”.

Thus, another embodiment of the present invention describes a method for preventing and/or treating prion infections and/or diseases in an individual comprising the step of administering a pharmaceutically effective amount of at least one pharmaceutically active agent which activates at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1, or which activates or stimulates the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1. Preferably, said method is directed to the targets JNK2, PRK, PTP-SL, PTP-zeta, HSP86, and GPIR-1.

As used herein, the term “agent” or “pharmaceutically active agent” refers to any chemical compound capable of down- or upregulating, de- or increasing, suppressing, activation, stimulating or otherwise regulating the amount and/or activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1. Generally, said agents may be proteins, oligo- and polypeptides, nucleic acids, genes, aptamers, small chemical molecules, or other chemical moieties. An agent may be either an inhibitor or an activator and especially an inhibitor for the enzymes FGF-R1, Tkt, Abl, clk1, MKK7, LIMK-2, CaM-KI, and CDC 2 and an activator for the targets JNK2, PRK, PTP-SL, PTP-zeta, HSP86, and GPIR-1.

One special kind of said pharmaceutically active agents are aptamers which function as regulators of the activity of a wide range of cellular molecules such as human cellular protein kinase and phosphatase. Aptamers are nucleic acid molecules selected in vitro to bind small molecules, peptides, or proteins with high affinity and specificity. Aptamers not only exhibit highly specific molecular recognition properties but are also able to modulate the function of their cognate targets in a highly specific manner by agonistic or antagonistic mechanisms. Most famous examples for aptamers are DNA aptamers or RNA aptamers.

Further examples for pharmaceutically active agents are the pyridylpyrimidine compounds of the present invention and/or pharmaceutically acceptable salts thereof. Said compounds are administered in a dosage corresponding to an effective concentration in the range of 0.01-50 μM, preferably in the range of 0.01-10 μM, more preferably in the range of 0.01-1 μM, and most preferably in the range of 0.01-0.1 μM.

The compounds of general formula (I) can be administered in a daily dosage in the range of 25 mg to 1000 mg, preferably in a daily dosage of 400 mg to 600 mg, more preferably in a daily dosage of 500 mg, and most preferably in continuously increased daily dosages starting at a initial daily dosage of 400 mg and ending up in a daily dosage of 600 mg at the end of the treatment.

A question is how PrP^{c} does convert to PrP^{Sc}? Potential mechanisms that initiate conversion of PrP^{c} to PrP^{Sc} include a germ line mutation of the human prion protein gene (PRNP), a somatic mutation within a particular neuron, and spontaneous conversion of PrP^{c} to an aberrant conformation that is not refolded appropriately to its native structure. The prion protein gene (PRNP) is the single gene on the short arm of chromosome 20 in humans which encodes the normal cellular isoform of the prion protein. Regardless of the initiating event, once an “infectious unit” has been generated, PrP^{Sc} appears to act as a conformational template by which PrP^{c} is converted to a new molecule of PrP^{Sc} through protein-protein interaction of PrP^{Sc} and PrP^{c}. This concept is supported by several studies which show that mice with the normal PrP gene deleted (PrP knockout mice) do not develop prion disease after inoculation with scrapie. Furthermore, transgenic (Tg) mice that express a chimeric PrP gene made of human and mouse segments develop protease-resistant chimeric mouse-human Prp^{Sc} in their brains when inoculated with brain extracts from humans with prion disease. These findings clearly illustrate that prions do not self-replicate but instead convert nonpathogenic PrP^{c} to pathogenic PrP^{Sc}.

In its sporadic or nonfamilial form, CJD is the most common of the human prion diseases. Confusion and forgetfulness which progress rapidly to severe cortical dementia in combination with ataxia, myoclonus, and an abnormal electroencephalogram (EEG) represents the “classic tetrad” of CJD. However, a host of other neurologic signs and symptoms, including diffuse or focal weakness, painful neuropathy, chore-iform movements, hallucinations, cortical blindness, primary language disturbance, supranuclear ophthalmoplegia, and alien hand syndrome, among others, have been observed. As the disease progresses from the early stage, ataxia commonly limits the patient's mobility.

Familial CJD (fCJD) includes those cases with a dominantly inherited mutation of the PRNP gene, in which the pathologic features of spongiform change occur in the absence of GSS-type plaques. Although, familial cases of CJD tend to have a clinical and pathologic phenotype similar to that of sCJD.

The original description of a patient with the onset of ataxia and dysarthria followed by variable degrees of pyramidal and extrapyramidal symptoms and late developing dementia defines the classic presentation of GSS. The duration of said disease ranges from 2 to 10 years. Death usually results from secondary infection, often from aspiration pneumonia because of impaired swallowing. The presence of plaque deposits regionally or diffusely throughout the cortex that are immunoreactive to anti-human PrP antibodies is the hallmark of this form of prion disease.

FFI is a genetic disorder which manifests itself by many symptoms due to the degeneration of a certain part of the brain, the thalamus. The affected area of the brain is the area responsible for sleep, the thalamus. The thalamus is the center which communications from the brain to the body and the body to the brain pass through for proper directions to where a signal should be received. When sleep takes place, it is thought that the thalamus becomes less efficient at this signal transfer function allowing for the vegetative state of sleep to come over an individual. Consequently, the symptoms of fatal familial insomnia are directly related to the malfunction of the responsibilities of the thalamus, namely sleep.

There are four stages of the disease before an individual's life ends. The first stage is progressive insomnia, the characteristic feature of fatal familial insomnia. By now, there is no cure for this illness.

The term “familial” means: affecting several members of the same family, usually as a result of an underlylng genetic mutation.

The occurrence of vCJD is sobering because it appears to represent a situation in which the prion has “jumped” species, in this case from cow to human. Because the pathologic features and clinical presentation of vCJD differ significantly from those of sCJD, it is considered a new “strain” of human prion disease. The same “protein signature” was observed following experimental transmission of BSE to several animal hosts, supporting the idea that vCJD results from the infection of humans with BSE. vCJD occurs primarily in younger individuals (average age 27) with a somewhat protracted course of approximately 16 months. The brain shows diffuse vacuolation and the presence of distinctive dense core PrP-containing plaques surrounded by a halo of spongiform change.

Kuru is the condition which first brought prion diseases to prominence in the 1950s. The disease was found in geographically isolated tribes in New Guinea. It was established that ingesting brain tissue of dead relatives for religious reasons was likely to be the route of transmission.

Alpers Syndrome is the name given to prion diseases in infants.

Scrapie is the accepted, albeit somewhat colloquial, name for the naturally occurring transmissible spongiform encephalopathy of sheep and goats found worldwide. Scrapie also infects laboratory mice and hamsters making it one of the most important sources of new scientific information about this group of disorders. Scrapie was the first example of this type of disease to be noticed and has been known about for many hundreds of years. There are two possible methods of transmission in sheep: a) Infection of pasture with placental tissue carrying the agent followed by ingestion, or b) direct sheep-lamb transmission. CWD is a fatal neurodegenerative disease of deer and elk, now known to be a transmissible spongiform encephalopathy. To date, affected animals have been found exclusively in the United States.

BSE

Bovine spongiform encephalopathy or “mad cow disease” appears to have originated from scrapie that has been recognized in Europe since the mid-18th century. It has since spread to most sheep-breeding countries and is widespread in the United Kingdom, where until 1988 the rendered carcasses of livestock (including sheep) were fed to ruminants and other animals as a protein-rich nutritional supplement.

During rendering, carcasses from which all consumable parts had been removed were milled and then decomposed in large vats by boiling at atmospheric or higher pressures, producing an aqueous slurry of protein under a layer of fat (tallow). After the fat was removed, the slurry was desiccated into a meat and bone meal product that was packaged by the animal food industry and distributed to owners of livestock and other captive animals (e.g., zoo and laboratory animals, breeding species, pets).

A further aspect is related to a method for regulating the expression of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 in an individual comprising the step of administering the individual a pharmaceutically effective amount of at least one pharmaceutically active agent wherein said agent inhibits at least partially the transcription of DNA or the translation of RNA.

And a still further aspect of the present invention relates to a method for regulating the expression of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 in the cells, the method comprising the step of administering the cells a pharmaceutically effective amount of at least one pharmaceutically active agent wherein said agent inhibits at least partially the transcription of DNA or the translation of RNA.

As used herein, the term “regulating expression and/or activity” generally refers to any process that functions to control or modulate the quantity or activity (functionality) of a cellular component. Static regulation maintains expression and/or activity at some given level. Upregulation refers to a relative increase in expression and/or activity. Accordingly downregulation refers to a relative decrease in expression and/or activity. Downregulation is synonymous with inhibition of a given cellular component's activity.

The transcription of DNA and the translation of RNA can be inhibited by oligonucleotides or oligonucleotide derivatives. Thus, the present invention discloses oligonucleotides and derivatives of oligonucleotides which may be used in the above-mentioned methods. The oligonucleotide and/or its derivatives bind to the DNA and/or RNA encoding a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 and suppress the transcription of DNA or translation of RNA.

As described above, said prion infection and/or disease associated with said prion infection is selected from the group comprising Scrapie, TME, CWD, BSE, vCJD, CJD, GSS, FFI, Kuru, and Alpers Syndrome. Preferably, the method is used for prophylaxis and/or treatment of BSE, vCJD, or CJD. The above disclosed methods are preferably applied to CJD, vCJD, and BSE, more preferably applied to vCJD and BSE, and most perferably applied to BSE.

Some methods of the present invention identify compounds useful for prophylaxis and/or treatment of prion infections and/or diseases by screening a test compound, or a library of test compounds; for its ability to inhibit at least one of the above-mentioned human cellular protein kinases, phosphatases, or cellular signal transduction molecules, identified herein as characteristically up- or downregulated during prion production or growth inside a cell or individual. A variety of assay protocols and detection techniques are well known in the art and easily adapted for this purpose by a skilled practitioner. Such methods include, but are not limited to, high throughput assays (e.g., microarray technology, phage display technology), and in vitro and in vivo cellular and tissue assays.

Thus, a solid support is disclosed in the present invention useful for screening compounds useful for the prophylaxis and/or treatment of prion infections and/or diseases in an individual, the solid support comprising at least one immobilized oligonucleotide, wherein said oligonucleotide encodes one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

A further aspect of the present invention is related to a solid support useful for screening compounds useful for the prophylaxis and/or treatment of prion infections and/or diseases in an individual, the solid support comprising at least one immobilized human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

In another embodiment, a component of the above-mentioned methods comprises peptide fragments of one or more of the above-identified human cellular protein kinases, phosphatases or cellular signal transduction molecules immobilized on a solid support. Once again the most preferred solid support embodiment would contain polymers of sufficient quality and quantity to detect all of the above-mentioned human cellular protein kinases, phosphatase and cellular signal transduction molecules (e.g., a nucleic acid or a peptide microarray). A variety of supports and constructions of the same for the methods disclosed herein are well known in the art and easily adapted for this purpose by a skilled practitioner (cf., for example: Marschall, 1999 “Do-it-yourself gene watching” Science 286, 444-447; Service 2000 “Protein arrays step out of DNA's shadow” Science 289, 1673).

It is preferred that mRNA is measured as an indication of expression. Methods for assaying for mRNA include, but are not limited to, Northern blots, slot blots, dot blots, and hybridization to an ordered array of oligonucleotides. Nucleic acid probes useful for assay of a sample are preferably of sufficient length to specifically hybridize only to appropriate, complementary transcripts. Typically the oligonucleotide probes will be at least 10 to 25 nucleotides in length. In some cases longer probes of at least 30 to 50 nucleotides will be desirable.

The cDNA oligonucleotides immobilized on said membrane filter which are used for detecting the up- or downregulation of the above-mentioned human cellular protein kinases, phosphatases, and cellular signal transduction molecules by hybridization to the radioactively labeled cDNA probes have the nucleotide sequences listed in table 1.

TABLE 1
Nucleotide sequences of cDNA-arrays

| | Sequence of immobilized DNA | |
|---|---|---|
| Cellular kinase, phosphatase, or | on arrays (in relation to the | |
| signal transduction molecule | respective Acc No) | |
| FGF-R1 | 41 bp-2619 bp | (X52833) |
| Tkt (EC 2.7.1.112) | 1 bp-3096 bp | (X74764) |
| Abl | 2153 bp-3765 bp | (M14752) |
| clk1 | 156 bp-1610 bp | (L29219) |
| MKK7 | 77 bp-1323 bp | (AF013588) |
| CDC2 | 77 bp-1050 bp | (X05360) |
| CaMKI | 145 bp-1452 bp | (L41816) |
| JNK2 | 507 bp-1782 bp | (L31951) |
| LIMK-2 | 963 bp-2047 bp | (D45906) |
| PRK | n.a bp-1862 bp | (U56998) |
| PTP zeta (EC 3.1.3.48) | 148 bp-7604 bp | (X54135) |
| PTP-SL | 862 bp-1902 bp | (NM_002849) |
| HSP86 | n.a bp—n.a bp | (X07270) |
| GPIR-1 | n.a bp—n.a bp | (n.a) |

Tkt has been assigned to the EC Number: 2.7.1.112

PTP zeta has been assigned to the EC Number: 3.1.3.48

The nucleoside sequences of the genes coding for the human cellular protein kinases, phosphatases, or cellular signal transduction molecules listed in Table 1 together with the amino acid sequences and the enzyme commission numbers (E.C. numbers) of said enzymes can be obtained from NCBI (National Library of Medicine: PubMed; Web address: www.ncbi.nlm.nih.gov/entrez).

The polypeptide product of gene expression may be assayed to determine the amount of expression as well. Methods for assaying for a protein include, but are not limited to, western blot, immuno-precipitation, radioimmuno assay, and peptide immobilization in an ordered array. It is understood, however, that any method for specifically and quantitatively measuring a specific protein or mRNA product can be used.

A variety of supports upon which nucleic acids or peptides can be immobilized are known in the art, for example filters, or polyvinyl chloride dishes. Any solid surface to which oligonucleotides or peptides can be bound, either directly or indirectly, either covalently or non-covalently, can be used. A preferred solid support is a microarray membrane filter or a “biochip”. These contain particular polymer probes in predetermined locations on the array. Each predetermined location may contain more than one molecule of the probe, but each molecule within the predetermined location has an identical sequence.

The present invention incorporates by reference in their entirety techniques well known in the field of molecular biology. These techniques include, but are not limited to, techniques described in the following publications:

Ausubel, F. M. et al. eds., “Short Protocols In Molecular Biology” 4^{th} Ed. 1999, John Wiley & Sons, NY (ISBN 0-471-32938-X);

Old, R. W. & S. B. Primrose “Principles of Gene Manipulation: An Introduction To Genetic Engineering” 3^{rd} Ed. 1985, Blackwell Scientific Publications, Boston. Studies in Microbiology: V.2, 409 pp. (ISBN 0-632-01318-4);

Mayer, R. J. & J. H. Walker eds. “Immunochemical Methods In Cell and Molecular Biology” 1987, Academic Press, London. 325 pp. (ISBN 0-12480-855-7);

Winnacker, E. L. “From Genes To Clones: Introduction To Gene Technology” 1987 VCH Publishers, NY. (translated by Horst Ibelgaufts) 634 pp. (ISBN 0-89573-614-4).

As described above, a microarray platform technology was developed consisting of more than 1100 signal transduction cDNAs immobilized on a solid support. Thus, another aspect of the present invention is directed to a solid support useful for detecting prion infections and/or diseases in an individual, the solid support comprising an immobilized oligonucleotide, wherein said oligonucleotide is capable of detecting activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

The present invention discloses also for the first time a solid support useful for detecting prion infections and/or diseases in cells, the solid support comprising an immobilized oligonucleotide, wherein said oligonucleotide is capable of detecting activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

The present invention further incorporates by reference in their entirety techniques well known in the field of microarray construction and analysis. These techniques include, but are not limited to, techniques described in the following patents and patent applications describing array of biopolymeric compounds and methods for their fabrication:

U.S. Pat. Nos. 5,807,522; 6,087,102; WO 93/17126; WO 95/11995; WO 95/35505; EP 742 287; and EP 799 897.

Techniques also include, but are not limited to, techniques described in the following patents and patent application describing methods of using arrays in various applications;

U.S. Pat. Nos. 5,994,076; 6,033,860; 6,040,138; 6,040,140; WO 95/21265; WO 96/31622; WO 97/10365; WO 97/27317; EP 373 203; and EP 785 280

Still a further aspect of the present invention is directed to pharmaceutical compositions comprising at least one pharmaceutically active agent together with a pharmaceutically acceptable carrier, excipient or diluents. Examples for pharmaceutically active agents are the above-mentioned inventive compounds according to formula (I), or other small chemical molecules, antibodies, aptamers, oligo- and polynucleotides, genes and other biological components capable of regulating the activity of at least one target selected from the group comprising FGF-R1, Tkt, Abl, clk1, MKK7, LIMK-2, CaM-KI, JNK2, CDC2, PRK, PTP-SL, PTP-zeta, HSP86, and GPIR-1, or which are effective to treat prion infections and diseases associated with prion infection. Said prion infections and diseases are preferably Scrapie, TME, CWD, BSE, vCJD, CJD, GSS, FFI, Kuru, and Alpers Syndrome.

Thus, the pharmaceutical compositions according to the present invention may comprise an inhibitor, such as the inventive pyridylpyrimidine compounds or an activator such as aptamers for at least one target selected from FGF-R1, Tkt, Abl, clk1, MKK7, LIMK-2, CaM-KI, JNK2, CDC2, PRK, PTP-SL, PTP-zeta, HSP86, and GPIR-1. It is also possible to have a combination of inhibitors or activators as active ingredients in one single pharmaceutical composition. Furthermore, suitable are also combinations of at least one inhibitor and at least one activator for different targets within a single pharmaceutical composition. For example, a pharmaceutical composition could comprise compound 12 as an inhibitor for, for instance, the target Abl, and an activator such as an aptamer for, for instance, the human cellular protein kinase JNK2.

Said pharmaceutical compositions are useful for the prophylaxis and/or treatment of an individual afflicted with prions comprising at least one agent capable of inhibiting and/or activating at least partially the activity, the expression, and/or the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

The pyridylpyrimidine compounds of the present invention are basic and form pharmaceutically acceptable salts with organic and inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, α-toluic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

It is also possible to obtain acid addition salts with amino acids like methionine, tryptophane, lysine or arginine, especially with pyridylpyrimidine compounds of the general formula (I) carrying a carboxylic acid residue.

Depending upon the substituents on the inventive pyridylpyrimidine compounds, one may be able to form salts with bases, too. Thus, for example, if there are carboxylic acid substituents in the molecule, salts may be formed with inorganic as well as organic bases such as, for example, NaOH, KOH, NH₄OH, tetraalkylammonium hydroxide, and the like.

The compounds of the general formula (I) can also be administered in form of their pharmaceutically active salts optionally using substantially nontoxic pharmaceutically acceptable carriers, excipients or diluents. The medications of the present invention are prepared in a conventional solid or liquid carrier or diluents and a conventional pharmaceutically-made adjuvant at suitable dosage level in a known way. The preferred preparations are in administratable form which is suitable for oral application. These administratable forms, for example, include pills, tablets, film tablets, coated tablets, capsules, powders and deposits.

The preferred administratable forms are tablets, film tablets, coated tablets, gelatin capsules, and opaque capsules. Each pharmaceutical composition contains at least one compound of the general formula (I), preferably compound 53 and/or pharmaceutically acceptable salts thereof in an amount of 50 mg to 150 mg, preferably 80 mg to 120 mg, and most preferably in an amount of 100 mg per formulation.

Furthermore, the subject of the present invention also includes pharmaceutical preparations for parenteral, including dermal, intradermal, intragastrical, intracutaneous, intravasal, intravenous, intramuscular, intraperitoneal, intranasal, intravaginal, intrabuccal, percutaneous, rectal, subcutaneous, sublingual, topical or transdermal application, which in addition to typical vehicles and diluents contain a pyridylpyrimidine compound of the general formula (I) and/or a pharmaceutically acceptable salt thereof as active ingredient.

Within the disclosed methods the pharmaceutical compositions of the present invention, containing pyridylpyrimidine derivatives of the general formula (I) as active ingredients, will typically be administered in admixture with suitable carrier materials selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

Suitable binders include starch, gelatin, natural sugars, corn sweeteners, natural and synthetic gums such as acacia, sodium alginate, carboxymethylcellulose, polyethylene glycol and waxes. Among the lubricants, there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. antihistaminic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidifies.

Also included are solid form preparations which are intended to be converted, shortly before use, to liquid form preparations for either oral or parenteral administration. Such liquid forms include solutions, suspensions and emulsions.

The inventive pyridylpyrimidine compounds of the present invention may also be deliverable transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refers to the active ingredients dispersed or solubilized in a hydrophillic semi-solid matrix.

Powders for constitution refers to powder blends containing the active ingredients and suitable diluents which can be suspended in water or juices.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluents in the composition can range from about 5 to about 95% by weight of the total composition, preferably from about 25 to about 75%, more preferably from about 30 to about 60% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 2 to about 20% by weight of the composition, more preferably from about 5 to about 10% by weight.

Binders characterize substances that bind or “glue” powders together and make them cohesive by forming granules, thus serving as the “adhesive” in the formulation. Binders add cohesive strength already available in the diluents or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and D,L-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents are materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

As used herein, a “pharmaceutically effective amount” of an inhibitor and/or an activator is an amount effective to achieve the desired physiological result, either in cells treated in vitro or in a subject treated in vivo. Specifically, a pharmaceutically effective amount is an amount sufficient to inhibit and or activate, for some period of time, one or more of the clinically defined pathological processes associated with the prion infection. The effective amount may vary depending on the specific inhibitor and/or activator selected, and is also dependent on a variety of factors and conditions related to the subject to be treated and the severity of the infection. For example, if an inhibitor and/or activator is to be administered in vivo, factors such as the age, weight and health of the patient as well as dose response curves and toxicity data obtained in pre-clinical animal work would be among those considered. If the inhibitor and/or activator is to be contacted with the cells in vitro, one would also design a variety of pre-clinical in vitro studies to assess such parameters as uptake, half-life, dose, toxicity, etc. The determination of a pharmaceutically effective amount for a given pharmaceutically active agent is well within the ability of those skilled in the art.

It is also apparent to a person skilled in the art that detection includes any method known in the art useful to indicate the presence, absence, or amount of a detection target. Such methods may include, but are not limited to, any molecular or cellular techniques, used singularly or in combination, including, but not limited to: hybridization and/or binding techniques, including blotting techniques and immunoassays; labeling techniques (chemiluminescent, colorimetric, fluorescent, radioisotopic); spectroscopic techniques; separations technology, including precipitations, electrophoresis, chromatography, centrifugation, ultrafiltration, cell sorting; and enzymatic manipulations (e.g., digestion).

It should be stressed that all above-mentioned features, aspects, and details of the present invention discussed and described in connection with infections and infectious diseases, equally apply to neurodegenerative diseases, like Alzheimer.

It is readily apparent to those skilled in the art that other suitable modifications and adaptations of the compositions and methods of the invention described herein are evident and may be made without departing from the scope of the invention or the embodiments disclosed herein. Having now described the present invention in detail, the same will be more clearly understood by reference to the following examples, which are included for purposes of illustration only and are not intended to be limiting of the invention.

### DESCRIPTION OF FIGURES

FIG. 1 shows 6 selected pyridylpyrimidine derivatives which are suitable inhibitors for prion diseases, namely compounds 4, 5, 37, 52, 84, and 88;

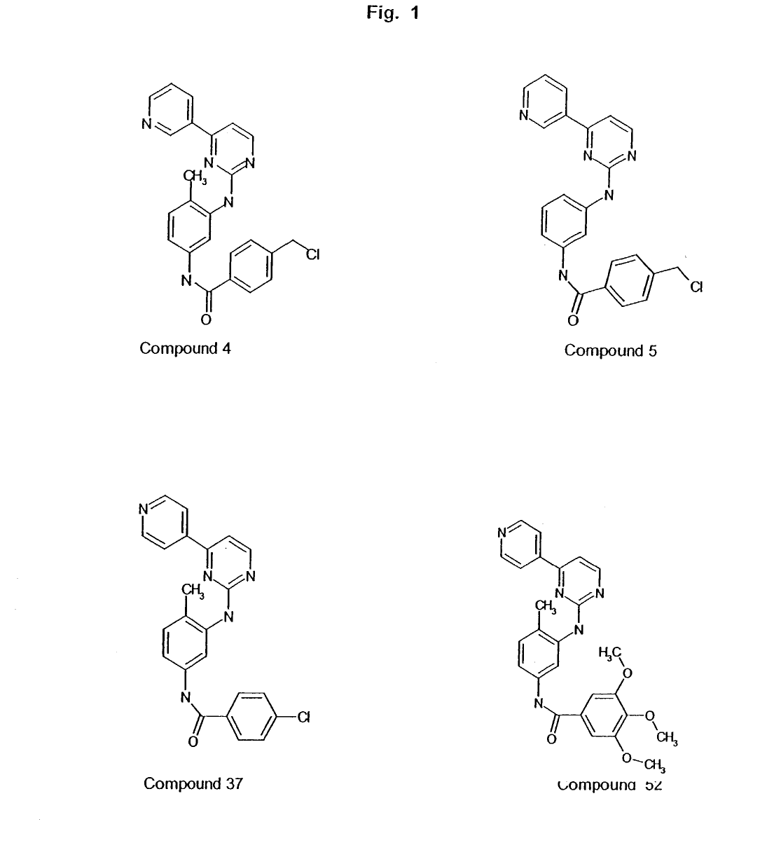

FIG. 2 shows the compound 4-(4-Methylpiperazin-1-ylmethyl)-N-[4-methyl-3-4-pyridin-3-yl-pyrimidin-2-ylamino)-phenyl]-benzamide, also known as Gleevec™;

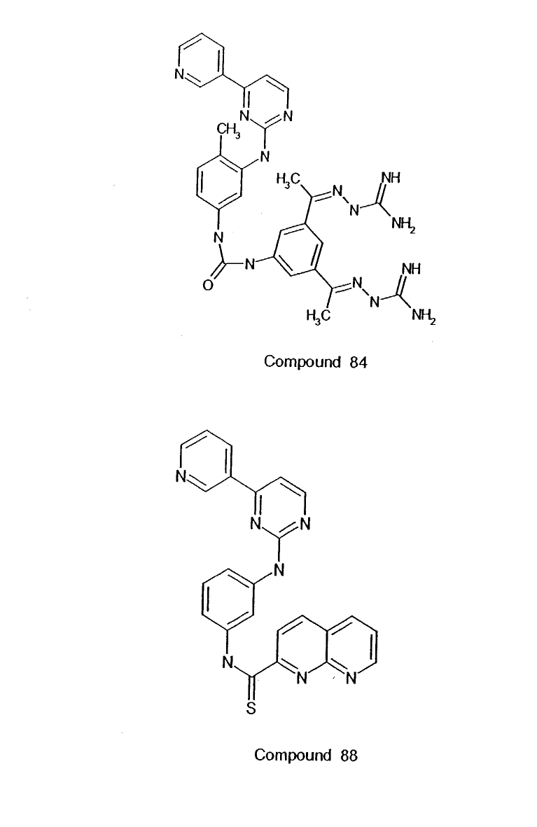

FIG. 3 shows selected compounds that have been identified as potent inhibitors in a prion propagation assay at a concentration of 5 μm.

### EXAMPLES

Materials and Methods

1. Generation of cDNA-Arrays on Membranes

In order to manufacture cDNAs-arrays on membranes, the following strategy was pursued: cDNAs encoding parts of or full length proteins of interest—in the following referred to as “target cDNAs”—were cloned into the plasmid Bluescript II KS⁺ (Stratagene, USA). Large scale purifications of these plasmids were performed according to standard techniques and 200 μl aliquots (1 μg/μl plasmid concentration) were transferred into appropriate 96well plates. Plates were closed with sealing tape and chilled on ice for 5 minutes after incubation for 10 minutes at 95° C. 10 μl of 0.6 N NaOH were added and the mix was stored for 20 minutes at room temperature before addition of 10 μl 2.5 M Tris-HCl pH 7.1 and 20 μl 40×SSC (3 M NaCl, 300 mM Sodium Citrate, pH 7.0). Target cDNAs were spotted onto Nylon or Nitrocellulose membranes using a BioGrid (BioRobotics, UK) equipped with a 0.7 mm pintool. In this way, between 200 ng and 350 ng of plasmids encoding target cDNAs were transferred onto the membranes and crosslinked to the membranes by ultraviolet light (1.2×10⁵ μJ/cm²). The arrays were stored for use in subsequent experiments at room temperature.

2. Generation of Cells

PrP^{Sc}- and PrP^{c}-transfected mouse neuronal cells (N2A) were cultured in MEM (Minimum Essential Medium, Life Technologies) supplemented with 10% fetal calf serum at 37° C. and 5% CO₂ to obtain ˜6×10⁶ cells per tissue culture flask.

3. Lysis of Cells, Isolation of Total RNA and Purification of PolyA⁺ RNA

After incubation of the cells with the virus for the respective time-points, cells were washed twice with phosphate buffered saline (PBS) and then trypsinized. Subsequently, cells were removed from the culture dish by resuspension with PBS. Afterwards, cells were sedimented and directly lysed in Tri reagent by repetitive pipetting using in 1 ml of Tri reagent (Molecular Research Centre, Inc., USA) per 1×10⁶ cells.

The lysates were stored at room temperature for 5 minutes and then centrifuged at 12000×g for 15 minutes at 4° C. The supernatant was mixed with 0,1 ml of 1-bromo-3-chloropropane per 1 ml of Tri reagent and vigorously shaken. The suspension was stored for 5 minutes at room temperature and then centrifuged at 12000×g for 15 minutes at 4° C.

The colourless upper phase was transferred into new tubes, mixed with 5 μl of poly-acryl-carrier (Molecular Research Centre, Inc., USA) and with 0.5 ml of isopropanol per 1 ml of Tri reagent and vigorously shaken. The samples were stored at room temperature for 5 minutes and then centrifuged at 12000×g for 8 minutes at 4° C. The supernatant was removed and the RNA pellet washed twice with 1 ml of 75% ethanol. The pellet was dried and resuspended for 10 minutes at 55° C. in 50 μl of RNase-free buffer (5 mM Tris-HCl pH 7.5). The integrity of the isolated RNA was determined by agarose/formaldehyde gel electrophoresis and the RNA was finally stored at −70° C. for use in subsequent experiments.

4. Preparation of Radioactively Labelled cDNA Probes from RNA

In order to obtain radioactively labelled cDNA probes total RNA was transcribed into a cDNA-probe in the presence of radioactively labelled dATP. 12 μl bidestilled DEPC (Diethylpyrocarbonate) treated H₂O containing 0.5 μg of primer TXN (5′TTT TTT TTT TTT TTT TXN-3′ with T→dTTP; N→dATP, dCTP, dGTP or dTTP; X→dATP, dCTP or dGTP) and total RNA (1 to 10 μg) were shaken between 5 and 15′ at 60° C. and then incubated on ice for 2 minutes. After centrifugation (30 seconds, 10000×g) 7 μl of a mix consisting of 100 μCi dATP-P³³ (Amersham, UK) which were dried under vacuum previously and resuspended in 4 μl first strand buffer (Life Technologies, USA), 2 μl 0.1M DTT (Dithiothreitol) and 1 μl labelling solution (4 mM dCTP, dGTP, dTTP each and 80 μM dATP final concentration) were added. Following the addition of 1 μl Superscript II reverse transcriptase (Life Technologies, USA) the reaction was incubated for 10 minutes at room temperature and then for 60 minutes at 38° C. Subsequently, the reaction was vigorously shaken for 30 minutes at 68° C. after adding 5 μl 0.5 M EDTA and 25 μl 0.6M NaOH.

Unincorporated nucleotides were removed from the labelling reaction using ProbeQuant G-50 columns (Amersham, UK). The column was vigorously shaken and centrifuged for 1 minute at 735×g in an appropriate reaction tube after bottom closure and lid were removed. The column was placed into a new reaction tube, the probe was applied onto the centre of the column material and the column was centrifuged for 2 minutes at 735×g. The flow-trough was transferred into new reaction tubes and filled up to a volume of 100 μl with bidestilled H₂O. The probe was precipitated by centrifugation for 15 minutes at 12000×g after 4 μl 5M NaCl, 1 μl poly-acryl-carrier (Molecular Research Centre, Inc., USA) and 250 μl ethanol were added. The supernatant was discarded and the pellet was dried at 50° C. for 5 minutes before starting with the hybridisation.

5. Hybridisation of Radioactively Labelled cDNA-Probes to cDNA-Arrays

The pellet was resuspended in 10 μl C₀T DNA (1 μg/μl, Roche Diagnostics, Germany), 10 μl yeast tRNA (1 μg/μl Sigma, USA) and 10 μl polyA (1 μg/μl, Roche Diagnostics, Germany) and incubated at 55° C. for 5 minutes. Herring sperm DNA was added to a final concentration of 100 μg/ml and the volume was filled up to 100 μl with 5 μl 10% SDS (Sodiumdodecylsulfat), 25 μl 20×SSPE (3M Sodium chloride, 0,2 M Sodium dihydrogen phosphate monohydrate, 0,02 M Ethylenedinitrilo tetraacetic acid, disodium salt dihydrate; pH 7,4 ) and bidestilled H₂O. The mix was put on 95° C. for 5 minutes, centrifuged for 30 seconds at 10000×g and vigorously shaken for 60 minutes at 65° C. A 1 μl aliquot of the probe was used to measure the incorporation of radioactive dATP with a scintillation counter. Probes with at least a total of 20×10⁶ cpm were used.

The arrays were prehybridised for at least 3 hours at 42° C. in hybridisation solution in a roller bottle oven. After prehybridization the radioactively labelled probe was added into the hybridisation solution and hybridisation was continued for 20 to 40 hours.

The probe was discarded and replaced with wash solution A (2×SSC). The arrays were washed twice in wash solution A at room temperature in the roller oven. Afterwards, wash solution A was replaced by wash solution B (2×SSC, 0.5% SDS) preheated to 65° C. and arrays were washed twice for 30 minutes at 65° C. Then, wash solution B was replaced by wash solution C (0.5×SSC, 0.5% SDS) preheated to 65° C. and arrays were washed twice for 30 minutes at 65° C. The moist arrays were wrapped in airtight bags and exposed for 8 to 72 hours on erased phosphoimager screens (Fujifilm, Japan).

6. Analysis of cDNA-Arrays

The exposed phosphoimager screens were scanned with a resolution of 100μ and 16bits per pixel using a BAS-1800 (Fujifilm,,Japan). Files were imported into the computer program ArrayVision (Imaging Research, Canada). Using the program's features, the hybridization signals of each target cDNA were converted into numbers. The strength of the hybridization signals reflected the quantity of RNA molecules present in the probe. Differentially expressed genes were selected according to the ratio of their signal strength after normalization to the overall intensity of the arrays.

7. Cell Culture and Expression of 3F4-Tagged PrP (3F4-ScN2a)

The mouse neuroblastoma cell line 3F4-ScN2a represents a stably transfected clone of ScN2a cells (PrP^{Sc} infected N2a cells) which overexpress 3F4-epitope-tagged murine PrP. Residues 109 and 112 of murine PrP were replaced by methionine to introduce the epitope for reactivity with the monoclonal anti-PrP antibody 3F4. Cells were maintained in Dulbecco's modified Eagle's (DMEM) or Opti-MEM medium containing 10% fetal calf serum, antibiotics and glutamin. For generation of stable transfectants we used the vector pcDNA3.1/Zeo (Invitrogen; Leek, The Netherlands). Lipofection of cells with recombinant plasmids was done using standard procedures and recombinant clones were selected by addition of 300 μg Zeocin/ml medium.

8. Treatment of Cells with Inhibitors

All tested compounds were solubilized in DMSO (dimethylsulfoxide), and prepared as 10 mM stock solutions. The drugs were applied to the cells described above for three days in final concentrations between 5 and 20 μM.

9. Immunoblot and Proteinase K (PK) Analysis

Confluent cell cultures were lysed in cold lysis buffer (10 mM Tris-HCl, pH 7.5; 100 mM NaCl; 10 mM EDTA; 0.5% Triton X-100; 0.5% DOC) (EDTA: ethylene diamine tetraacetate; Triton X-100: t-octylphenoxypolyethoxyethanol; DOC: deoxycholic acid). Postnuclear lysates were split between those with and without proteinase K digestion. Samples without proteinase K digestion were supplemented with proteinase inhibitors (5 mM PMSF, 0.5 mM Pefabloc, and aprotinin) (PMSF: phenylmethylsulfonyl fluoride) and directly precipitated with ethanol. Samples for proteinase K digestion were incubated with 20 μg/ml proteinase K for 30 min at 37° C.; digestion was stopped with proteinase inhibitors, and samples were ethanol precipitated. After centrifuging for 30 min at 3,500 rpm the pellets were redissolved in TNE buffer (10 mM Tris-HCl pH7.5, 100 mM NaCl, 1 mM EDTA) and gel loading buffer was then added. After boiling for 5 min an aliquot was analyzed on 12.5% PAGE. For Western blot analysis, the proteins were electrotransferred to PVDF membranes (polyvinylidendifluorid). The membrane was blocked with 5% non-fat dry milk in TBST (0.05% Tween 20, 100 mM NaCl, 10 mM Tris-HCl, pH 7.8) (Tween 20: polyoxyethylenesorbitan monolaurate; Tris-HCl: Tris-(hydroxymethyl)-aminomethane-hydrochloride), incubated overnight with the primary antibody at 4° C. and stained using the enhanced chemiluminescence blotting kit from Amersham Corporation. Specific immuno-staining of the PrP^{c} and PrP^{Sc} forms were obtained with the prion protein specific antibody 3F4 (Signet Pathologies, U.S.A.).

10. Results

Determination of the amount of the pathogenic form of the prion protein PrP^{Sc} upon treatment of prion infected cells with different types of small molecule protein kinase inhibitors resulted in the identification of a compound class of pyridylpyrimidine derivatives examplified by the compound 4-(4-Methylpiperazin-1-ylmethyl)-N-[4-methyl-3-(4-pyridin-3-yl-pyrimidin-2-ylamino)-pheny]-benzamide (compound 53) and compounds 4, 5, and 37.

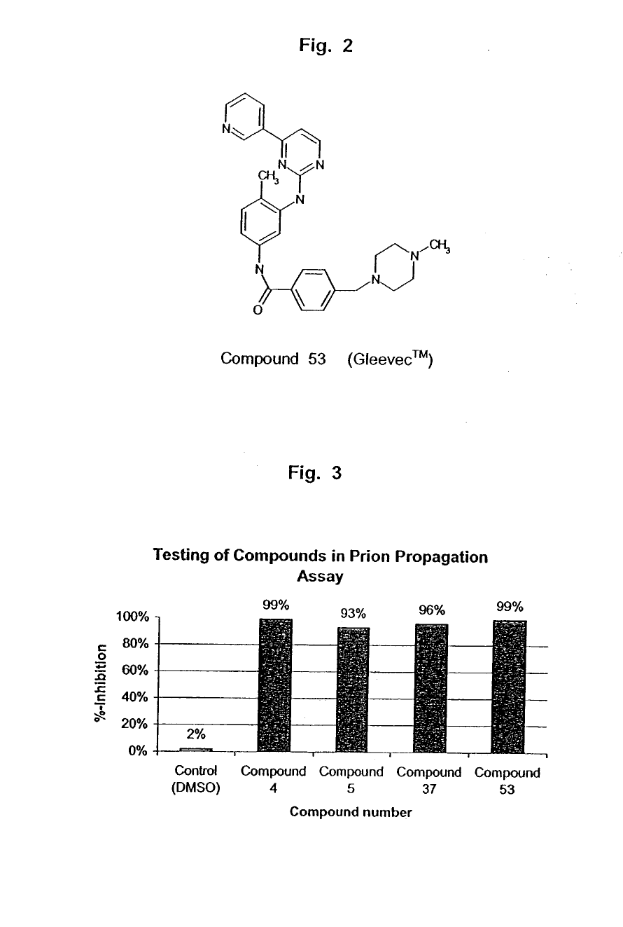

These compounds significantly reduced the amount of PrP^{Sc} in prion infected cells in a concentration range between 5 and 20 μM (final concentration). As shown in FIG. 3 the selected compounds 4, 5, 37, and 53 inhibit almost completely the activity of prion propagation within said concentration range.

The compounds did not show any toxic effects on the cells in these concentrations. Therefore these molecules described herein serve as potential inhibitors for the medical intervention of prion diseases such as transmissible spongiform encephalitis (TSE) infections which include Bovine spongiform encephalitis (BSE) or the new variant of Creutzfeld Jakob disease (vCJK).

1. Compounds having the general formula (I): 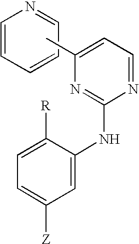
wherein:
R represents hydrogen or methyl;
Y, Y′, Y″ are independently of each other —H, —F, —Cl, —Br, —I, —CH₂F, —CH₂Cl, —CH₂Br, —CH₂I, —OH, —OCH₃, —CH₃, —CN, —OCF₃, 4-methylpiperazin-1-yl-methyl, —C(CH₃)═N—NH—C(NH)—NH₂;
Z represents —NO₂, —NH₂, —NH—CO—X, —NH—CS—X, —NH—CO—NH—X, —NH—SO₂—X;
X represents thiophenyl, cyclohexyl, isoquinolinyl, naphthyl, quinolinyl, cyclopentyl, pyridinyl, naphthyridinyl, or 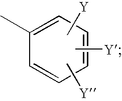
and pharmaceutically acceptable salts thereof.

2. Use of a compound having the general formula (I): 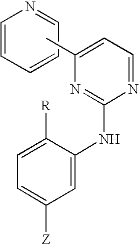
wherein:
R represents hydrogen or methyl;
Y, Y′, Y″ are independently of each other —H, —F, —Cl, —Br, —I, —CH₂F, —CH₂Cl, —CH₂Br, —CH₂I, —OH, —OCH₃, —CH₃, —CN, —OCF₃, 4-methylpiperazin-1-yl-methyl, —C(CH₃)═N—NH—C(NH)—NH₂;
Z represents —NO₂, —NH₂, —NH—CO—X, —NH—CS—X, —NH—CO—NH—X, —NH—SO₂—X;
X represents thiophenyl, cyclohexyl, isoquinolinyl, naphthyl, quinolinyl, cyclopentyl, pyridinyl, naphthyridinyl, or 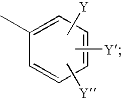
and pharmaceutically acceptable salts thereof as pharmaceutically active agents.

3. Use of a compound having the general formula (I): 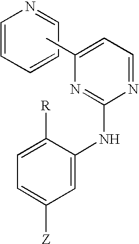
wherein:
R represents hydrogen or methyl;
Y, Y′, Y″ are independently of each other —H, —F, —Cl, —Br, —I, —CH₂F, —CH₂Cl, —CH₂Br, —CH₂I, —OH, —OCH₃, —CH₃, —CN, —OCF₃, 4-methylpiperazin-1-yl-methyl, —C(CH₃)═N—NH—C(NH)—NH₂;
Z represents —NO₂, —NH₂, —NH—CO—X, —NH—CS—X, —NH—CO—NH—X, —NH—SO₂—X;
X represents thiophenyl, cyclohexyl, isoquinolinyl, naphthyl, quinolinyl, cyclopentyl, pyridinyl, naphthyridinyl, or 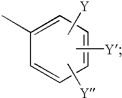
and pharmaceutically acceptable salts thereof for prophylaxis and/or treatment of infectious diseases or neurodegenerative diseases.

4. Use of a compound according to claim 2 or 3 for the prophylaxis and/or treatment of prion infections and/or diseases induced by prion infection.

5. Use of a compound according to any one of claims 2-4 wherein R represents hydrogen.

6. Use of a compound according to any one of claims 2-5 wherein Z represents —NH—CO—X or —NH—SO₂—X.

7. Use of a compound according to any one of claims 2-6 wherein Y, Y′, Y″ are independently of each other —H, —F, —Cl, —CH₂F, —CH₂Cl, —OH, —OCH₃, —CH₃, —CN, —OCF₃, 4-methylpiperazin-1-yl-methyl.

8. Use of a compound according to claim 2 or 3 wherein the compound is selected from the group comprising:
(3-Nitrophenyl)-(4-pyridin-3-yl-pyrimidin-2-yl)-amine;
(3-Aminophenyl)-(4-pyridin-3-yl-pyrimidin-2-yl)-amine;
(5-Amino-2-methylphenyl)-(4-pyridin-3-yl-pyrimidin-2-yl)-amine;
4-Chloromethyl-N-[4-methyl-3-(4-pyridin-3-yl-pyrimidin-2-ylamino)-phenyl]-benzamide;
4-Chloromethyl-N-[3-(4-pyridin-3-yl-pyrimidin-2-ylamino)-phenyl]-benzamide;
4-(4-Methylpiperazin-1-ylmethyl)-N-[3-(4-pyridin-3-yl-pyrimidin-2-ylamino)-phenyl]-benzamide;
Thiophene-3-carboxylic acid [4-methyl-3-(4-pyridin-4-yl-pyrimidin-2-ylamino)-pheny]-amide;
4-Chloro-N-[4-methyl-3-(4-pyridin-3-yl-pyrimidin-2-ylamino)-phenyl]-benzamide;
4-Chloro-N-[3-(4-pyridin-3-yl-pyrimidin-2-ylamino)-phenyl]-benzamide;
3,4,5-Trimethoxy-N-[3-(4-pyridin-3-yl-pyrimidin-2-ylamino)-phenyl]-benzamide;
4-Cyano-N-[3-(4-pyridin-3-yl-pyrimidin-2-ylamino)-phenyl]-benzamide;
4-Methoxy-N-[3-(4-pyridin-3-yl-pyrimidin-2-ylamino)-phenyl]-benzamide;
4-Chloro-N-[3-(4-pyridin-3-yl-pyrimidin-2-ylamino)-phenyl]-benzenesulfonamide;
Thiophene-3-carboxylic acid [3-(4-pyridin-3-yl-pyrimidin-2-ylamino)-phenyl]-amide;
3,5-Dimethoxy-N-[3-(4-pyridin-3-yl-pyrimidin-2-ylamino)-phenyl]-benzamide;
3,4,5-Trimethoxy-N-[4-methyl-3-(4-pyridin-3-yl-pyrimidin-2-ylamino)-phenyl]-benzamide;
4-Cyano-N-[4-methyl-3-(4-pyridin-3-yl-pyrimidin-2-ylamino)-phenyl]-benzamide;
4-Methoxy-N-[4-methyl-3-(4-pyridin-3-yl-pyrimidin-2-ylamino)-phenyl]-benzamide;
4-Chloro-N-[4-methyl-3-(4-pyridin-3-yl-pyrimidin-2-ylamino)-phenyl]-benzenesulfonamide;
Thiophene-3-carboxylic acid [4-methyl-3-(4-pyridin-3-yl-pyrimidin-2-ylamino)-phenyl]-amide;
3,5-Dimethoxy-N-[4-methyl-3-(4-pyridin-3-yl-pyrimidin-2-ylamino)-phenyl]-benzamide;
4-Trifluoromethoxy-N-[4-methyl-3-(4-pyridin-3-yl-pyrimidin-2-ylamino)-phenyl]-benzamide;
Cyclohexanecarboxylic acid [4-methyl-3-(4-pyridin-3-yl-pyrimidin-2-ylamino)-phenyl]-amide;
Cyclohexanecarboxylic acid [3-(4-pyridin-3-yl-pyrimidin-2-ylamino)-phenyl]-amide;
Isoquinoline-5-sulfonic acid [4-methyl-3-(4-pyridin-3-yl-pyrimidin-2-ylamino)-phenyl]-amide;
Isoquinoline-5-sulfonic acid [3-(4-pyridin-3-yl-pyrimidin-2-ylamino)-phenyl]-amide;
(5-Nitro-2-methylphenyl)-(4-pyridin-2-yl-pyrimidin-2-yl)-amine;
(5-Amino-2-methylphenyl)-(4-pyridin-2-yl-pyrimidin-2-yl)-amine;
3,4,5-Trimethoxy-N-[4-methyl-3-(4-pyridin-2-yl-pyrimidin-2-ylamino)-phenyl]-benzamide;
4-Cyano-N-[4-methyl-3-(4-pyridin-2-yl-pyrimidin-2-ylamino)-phenyl]-benzamide;
(3-Aminophenyl)-(4-pyridin-2-yl-pyrimidin-2-yl)-amine;
4-Chloro-N-[4-methyl-3-(4-pyridin-2-yl-pyrimidin-2-ylamino)-phenyl]-benzamide;
Cyclohexanecarboxylic acid [4-methyl-3-(4-pyridin-2-yl-pyrimidin-2-ylamino)-phenyl]-amide;
4-Cyano-N-[4-methyl-3-(4-pyridin-4-yl-pyrimidin-2-ylamino)-phenyl]-benzamide;
4-Chloro-N-[4-methyl-3-(4-pyridin-4-yl-pyrimidin-2-ylamino)-phenyl]-benzenesulfonamide;
4-Methoxy-N-[4-methyl-3-(4-pyridin-4-yl-pyrimidin-2-ylamino)-phenyl]-benzamide;
4-Chloro-N-[4-methyl-3-(4-pyridin-4-yl-pyrimidin-2-ylamino)-phenyl]-benzamide;
Cyclohexanecarboxylic acid [3-(4-pyridin-4-yl-pyrimidin-2-ylamino)-phenyl]-amide;
3,5-Dimethoxy-N-[3-(4-pyridin-4-yl-pyrimidin-2-ylamino)-phenyl]-benzamide; (5-Amino-2-methylphenyl)-(4-pyridin-4-pyrimidin-2-yl)-amine;
Thiophene-3-carboxylic acid [3-(4-pyridin-4-yl-pyrimidin-2-ylamino)-phenyl]-amide;
4-Chloro-N-[3-(4-pyridin-4-yl-pyrimidin-2-ylamino)-phenyl]-benzenesulfonamide;
4-Chloro-N-[3-(4-pyridin-4-yl-pyrimidin-2-ylamino)-phenyl]-benzamide;
(3-Aminophenyl)-(4-pyridin-4-yl-pyrimidin-2-yl)-amine;
(3-Nitrophenyl)-(4-pyridin-4-yl-pyrimidin-2-yl)-amine;
4-Trifluoromethoxy-N-[4-methyl-3-(4-pyridin-4-yl-pyrimidin-2-ylamino)-phenyl]-benzamide;
Isoquinoline-5-sulfonic acid [3-(4-pyridin-4-yl-pyrimidin-2-ylamino)-phenyl]-amide;
4-Methoxy-N-[3-(4-pyridin-4-yl-pyrimidin-2-ylamino)-phenyl]-benzamide;
4Cyano-N-[3-(4-pyridin-4-yl-pyrimidin-2-ylamino)-phenyl]-benzamide;
3,4,5-Trimethoxy-N-[3-(4-pyridin-4-yl-pyrimidin-2-ylamino)-phenyl]-benzamide;
3,5-Dimethoxy-N-[4-methyl-3-(4-pyridin-4-yl-pyrimidin-2-ylamino)-phenyl]-benzamide;
3,4,5-Trimethoxy-N-[4-methyl-3-(4-pyridin-4-yl-pyrimidin-2-ylamino)-phenyl]-benzamide;
4-(4-Methylpiperazin-1-ylmethyl)-N-[4-methyl-3-(4-pyridin-3-yl-pyrimidin-2-ylamino)-phenyl]-benzamide;
4-Methyl-N-[4-methyl-3-(4-pyridin-2-yl-pyrimidin-2-ylamino)-phenyl]-benzenesulfonamide;
4-Methoxy-N-[4-methyl-3-(4-pyridin-2-yl-pyrimidin-2-ylamino)-phenyl]-benzamide;
3,5-Dimethoxy-N-[4-methyl-3-(4-pyridin-2-yl-pyrimidin-2-ylamino)-phenyl]-benzamide;
Naphthalene-2-carboxylic acid [4-methyl-3-(4-pyridin-3-yl-pyrimidin-2-ylamino)-phenyl]-amide;
N-[3-(4-Pyridin-4-yl-pyrimidin-2-ylamino)-phenyl]-benzamide;
4-Chloro-N-[3-(4-pyridin-2-yl-pyrimidin-2-ylamino)-phenyl]-benzamide;
4-Methoxy-N-[3-(4-pyridin-2-yl-pyrimidin-2-ylamino)-phenyl]-benzamide;
4-Chloro-N-[3-(4-pyridin-2-yl-pyrimidin-2-ylamino)-phenyl]-benzenesulfonamide;
Thiophene-2-carboxylic acid 3-(4-pyridin-2-yl-pyrimidin-2-yl-amino)-phenyl]-amide;
Naphthalene-2-sulfonic-acid [3-(4-pyridin-2-yl-pyrimidin-2-yl-amino)-phenyl]-amide;
Isoquinoline-5-sulfonic-acid [3-(4-pyridin-2-yl-pyrimidin-2-yl-amino)-phenyl]-amide;
Cylopentanecarboxylic acid 3-(4-pyridin-2-yl-pyrimidin-2-yl-amino)-phenyl]-amide;
Naphthalene-2carboxylic acid [3-(4-pyridin-2-yl-pyrimidin-2-ylamino)-phenyl]-amide;
4-Cyano-N-[3-(4-pyridin-2-yl-pyrimidin-2-ylamino)-phenyl]-benzamide;
3,5-Dimethoxy-N-[3-(4-pyridin-2-yl-pyrimidin-2-ylamino)-phenyl]-benzamide;
4-Bromo-N-[3-(4-pyridin-2-yl-pyrimidin-2-ylamino)-phenyl]-benzamide;
4-Methyl-N-[3-(4-pyridin-2-yl-pyrimidin-2-ylamino)-phenyl]-benzamide;
4-Fluoro-N-3-(4-pyridin-2-yl-pyrimidin-2-ylamino)-phenyl]-benzenesulfonamide;
3,5-Dichloro-N-[3-(4-pyridin-2-yl-pyrimidin-2-ylamino)-phenyl]-benzamide;
N-[3-(4-Pyridin-2-yl-pyrimidin-2-ylamino)-phenyl]-benzamide;
4-Chloromethyl-N-[3-(4-pyridin-2-yl-pyrimidin-2-ylamino)-phenyl]-benzamide;
4-Methyl-N-3-(4-pyridin-2-yl-pyrimidin-2-ylamino)-phenyl]-benzenesulfonamide;
4-(4-Methylpiperazin-1-ylmethyl)-N-[3-(4-pyridin-2-yl-pyrimidin-2-ylamino)-phenyl]-benzamide;
Naphthalene-2-carboxylic acid [3-(4-pyridin-4-yl-pyrimidin-2-ylamino)-phenyl]-amide;
2-Methoxy-N-[4-methyl-3-(4-pyridin-3-yl-pyrimidin-2-ylamino)-phenyl]-benzamide;
2-Methoxy-N-[3-(4-pyridin-3-yl-pyrimidin-2-ylamino)-phenyl]-benzamide;
4-Methyl-N-[3-(4-pyridin-3-yl-pyrimidin-2-ylamino)-phenyl]-benzamide;
4-Methyl-N-[4-methyl-3-(4-pyridin-3-yl-pyrimidin-2-ylamino)-phenyl]-benzamide;
N-[4-Methyl-3-(4-pyridin-3-yl-pyrimidin-2-ylamino)-phenyl]-benzamide;
1(3,5-Diacetyl-phenyl)-3-[4-methyl-3-(4-pyridin-3-yl-pyrimidin-2-ylamino)-pheny]-urea;
1-{3,5-Bis-(amidinohydrazone)-phenyl}-3-[4-methyl-3-(4-pyridin-3-yl-pyrimidin-2-ylamino)-phenyl]-urea;
N-[4-Methyl-3-(4-pyridin-3-yl-pyrimidin-2-ylamino)-phenyl]-nicotinamide;
N-[3-(4-Pyridin-3-yl-pyrimidin-2-ylamino)-phenyl]-nicotinamide;
[1,8]Naphthyridine-2carboxylic acid [3-(4-pyridin-3-yl-pyrimidin-2-ylamino)-phenyl]-amide;
[1,8]Naphthyridine-2carbothioic acid [3-(4-pyridin-3-yl-pyrimidin-2-ylamino)-phenyl]-amide;
2-Methoxy-N-[3-(4-pyridin-4-yl-pyrimidin-2-ylamino)-phenyl]-benzamide;
4-Trifluoromethoxy-N-[3-(4-pyridin-4-yl-pyrimidin-2-ylamino)-phenyl]-benzamide;
4-Methyl-N-[3-(4-pyridin-4-yl-pyrimidin-2-ylamino)-phenyl]-benzamide;
and/or a pharmaceutically acceptable salt of these compounds.

9. Use according to claim 8 wherein the compound is 4-(4-Methylpiperazin-1-ylmethyl)-N-[4-methyl-3-(4-pyridin-3-yl-pyrimidin-2-ylamino)-phenyl]-benzamide.

10. Use of a compound recited in any one of claims 2-9 and/or pharmaceutically acceptable salts thereof for the manufacture of a pharmaceutical composition for prophylaxis and/or treatment of prion infections and/or diseases induced by prion infection and/or neurodegenerative diseases.

11. Use according to claim 4 or 10 wherein said prion infection and/or disease is selected from the group comprising Scrapie, TME, CWD, BSE, CJD, vCJD, GSS, FFI, Kuru, and Alpers Syndrome.

12. Use according to claim 11 wherein said prion infection is BSE, vCJD, or CJD.

13. Use of a compound recited in any one of claims 2-9 as an inhibitor for at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

14. Use of a compound according to any one of claims 2 to 13 wherein the compound of the general formula (I) and/or pharmaceutically acceptable salts thereof is administered in a dosage corresponding to an effective concentration in the range of 0.01-50 μM.

15. Pharmaceutical composition comprising at least one compound recited in any one of claims 2-9 as an active ingredient, together with one or more pharmaceutically acceptable carrier(s), excipient(s) or diluents.

16. Method for preventing and/or treating infections and/or diseases in an individual which comprises administering to the individual an amount of at least one compound recited in claims 2-9 and/or pharmaceutically acceptable salts thereof effective to prevent and/or treat said infections and/or diseases.

17. Method for preventing and/or treating prion infections and/or prion diseases induced by prion infections in an individual which comprises administering to the individual an amount of at least one compound recited in any one of claims 3 to 8 and/or pharmaceutically acceptable salts thereof effective to prevent and/or treat said prion infection and/or disease.

18. Method for preventing and/or treating prion infections and/or prion diseases induced by prion infections in an individual which comprises administering to the individual an amount of at least one compound recited in claim 8 and/or pharmaceutically acceptable salts thereof effective to prevent and/or treat said prion infection and/or disease.

19. Method for detecting prion infections and/or prion diseases in an individual comprising:
a) providing a sample from said individual;
b) adding to said sample a pharmaceutically effective amount of at least one pharmaceutically active agent; and
c) detecting activity in said sample of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

20. Method according to claim 19 wherein said sample comprises blood, milk, saliva, sputum, excrement, urine, spinal cord liquid, liquor, lachrymal gland liquid, biopsies and all other samples that can be taken from a living animal or human for diagnostic purposes.

21. Method for detecting prion infections and/or prion diseases in cells, cell cultures and/or cell lysates comprising:
a) providing said cells, cell cultures and/or cell lysates;
b) adding to said cells, cell cultures and/or cell lysates a pharmaceutically effective amount of at least one pharmaceutically active agent; and
c) detecting activity in said sample of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

22. Method for preventing and/or treating prion infections and/or prion diseases in an individual comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

23. Method for preventing and/or treating prion infections and/or prion diseases in cell or cell cultures comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

24. Method for regulating the production of prions in an individual comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

25. Method for regulating the production of prions in cells comprising the step of administering a pharmaceutically effective amount of at least one pharmaceutically active agent which inhibits at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1, or which inhibits at least partially the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1.

26. A monoclonal or polyclonal antibody that binds to a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

27. Method according to any one of claims 19-25, wherein the agent is a monoclonal or polyclonal antibody which binds to a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

28. Method according to any one of claims 19-25, wherein the agent is at least one compound of the general formula (I) and/or pharmaceutically acceptable salts thereof.

29. Method according to any one of claims 16-25, wherein the agent is 4-(4-Methylpiperazin-1-ylmethyl)-N-[4-methyl-3-(4-pyridin-3-yl-pyrimidin-2-yl-amino)-phenyl]-benzamide and/or pharmaceutically acceptable salts thereof.

30. Method according to claim 28 wherein the compound of the general formula (I) and/or pharmaceutically acceptable salts thereof is administered in a dosage corresponding to an effective concentration in the range of 0.01-50 μM.

31. Method for detecting compounds useful for the prophylaxis and/or treatment of prion infections and/or diseases comprising:
a) contacting a test compound with at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, GPIR-1; and
b) detecting the activity of said human cellular protein kinase, phosphatase or cellular signal transduction molecule.

32. Method for preventing and/or treating prion infections and/or diseases in an individual comprising the step of administering a pharmaceutically effective amount of at least one pharmaceutically active agent which activates at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1, or which activates or stimulates the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

33. Method for regulating the production of prions in an individual comprising the step of administering an individual a pharmaceutically effective amount of at least one pharmaceutically active agent wherein said agent activates at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1, or wherein said agent at least partially activates or stimulates the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

34. Method for regulating the production of prions in cells comprising the step of administering the cells a pharmaceutically effective amount of at least one pharmaceutically active agent wherein said agent activates at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 or wherein said agent at least partially activates or stimulates the production of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 in the cells.

35. Method for regulating the expression of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 in an individual comprising the step of administering the individual a pharmaceutically effective amount of at least one pharmaceutically active agent wherein said agent inhibits at least partially the transcription of DNA or the translation of RNA.

36. Method for regulating the expression of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1 in the cells comprising the step of administering the cells a pharmaceutically effective amount of at least one pharmaceutically active agent wherein said agent inhibits at least partially the transcription of DNA or the translation of RNA.

37. Oligonucleotide that binds to the DNA or RNA encoding a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

38. Method according to claim 22, 23, 24, 25, 35 or 36 wherein the agent is a oligonucleotide which binds to the DNA and/or RNA encoding a human cellular protein kinase, phosphatase or a cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

39. Method according to claims 16, 17, 18, 19, 22, 24, 32, 33, or 35 wherein said individual is a human or ruminant.

40. Method according to any one of claims 17, 18, 19, 21, 22, 23, 31, or 32 wherein said prion infection and/or prion disease is selected from the group comprising Scrapie, TME, CWD, BSE, vCJD, CJD, GSS, FFI, Kuru, and Alpers Syndrome.

41. Method according to claim 40 wherein said prion infection and/or prion disease is BSE, vCJD, or CJD.

42. A solid support useful for detecting prion infections and/or diseases in an individual, the solid support comprising an immobilized oligonucleotide, wherein said oligonucleotide is capable of detecting activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

43. A solid support useful for detecting prion infections and/or diseases in cells, the solid support comprising an immobilized oligonucleotide, wherein said oligonucleotide is capable of detecting activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

44. A solid support useful for screening compounds useful for the prophylaxis and/or treatment of prion infections and/or diseases in an individual, the solid support comprising at least one immobilized oligonucleotide, wherein said oligonucleotide encodes one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

45. A solid support useful for screening compounds useful for the prophylaxis and/or treatment of prion infections and/or diseases in an individual, the solid support comprising at least one immobilized human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

46. Composition useful for the prophylaxis and/or treatment of an individual afflicted with prions comprising at least one agent capable of inhibiting at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

47. Composition useful for the prophylaxis and/or treatment of an individual afflicted with prions comprising at least one agent capable of activating or stimulating at least partially the activity of at least one human cellular protein kinase, phosphatase or cellular signal transduction molecule selected from the group comprising FGF-R1, Tkt, Abl, clk1, MKK7, LIMK-2, CaM-KI, JNK2, CDC2, PRK, PTP-SL, PTP-zeta, HSP86, and GPIR-1.

48. Composition according claim 46 or 47, wherein the agent is at least one compound of the general formula (I) and/or pharmaceutically acceptable salts thereof.
